(19) United States
(12) Patent Application Publication
(10) Pub. No.: US 2011/0144137 A1
(43) Pub. Date: Jun. 16, 2011

(54) METHOD FOR PREVENTING OR ALLEVIATING THE NOXIOUS EFFECTS RESULTING FROM TOXICANT EXPOSURE

(75) Inventors: Sven-Eric Jordt, New Haven, CT (US); Bret Fajans Bessac, Kingsville, TX (US); Michael Sivula, Braintree, MA (US); Maria Brackmann, Berlin (DE)

(73) Assignee: YALE UNIVERSITY, New Haven, CT (US)

(21) Appl. No.: 12/988,098

(22) PCT Filed: May 7, 2009
(86) PCT No.: PCT/US2009/002857

§ 371 Date: Mar. 1, 2011

Related U.S. Application Data
(60) Provisional application No. 61/126,819, filed on May 7, 2008.

(51) Int. Cl. A61K 31/522; A61K 31/505; A61P 43/00

(52) U.S. Cl. 514/263.35; 514/274

(57) ABSTRACT

The present invention provides a method of using agents which can modulate TRPA1 function as counteragents to inhibit the physical effects of chemical irritants/toxicants when given prior to exposure or to lessen the physical effects when administered post exposure, and more specifically, to a method for counteracting the acute physical noxious effects of toxicants, including but not limited to, tear gases, chlorine, hydrogen peroxide, ammonia, phosgene, chloropicrin, isocyanates and mustard gas. Administering the counteragents counteracts pain, inflammation, lachrymation, blepharospasm, respiratory irritation and depression, airway mucus secretion, airway obstruction and injury, cough and incapacitation and cutaneous chemical injuries. Another embodiment provides a method of preventing or treating a disease or condition in a mammal, which disease or condition includes hypersensitivity to chemical stimuli, particularly in regards to inflammatory airway conditions, such as asthma, rhinitis, etc., by administering to the mammal a therapeutically effective amount of a compound that inhibits TRPA1 function, wherein the compound reduces the hypersensitivity and mediates the response to such chemical stimuli in the mammal. The invention also includes a kit containing the compound that inhibits the TRPA1 function as a counteracting agent for administration prior to or post exposure to prevent or limit the effects of the exposure.

### RELATED APPLICATIONS

This application claims the benefit of priority to U.S. provisional application Ser. No. 61/126,819, filed May 7, 2008, which is incorporated by reference in its entirety herein.

### FIELD OF THE INVENTION

The present invention relates to the use of TRPA1 inhibitors to inhibit the effects of toxic gases on a subject or patient and as the basis for a therapy for treating toxicant exposures and the secondary effects and conditions which occur from such exposures.

### BACKGROUND

The release of methyl isocyanate in Bhopal, India caused the worst industrial accident in history. Exposure to this among other industrial isocyanates induce lacrimation, pain, airway irritation and edema. Similar responses are elicited by exposure to chemicals used as tear gases. Despite frequent exposures, the biological targets of isocyanates, tear gases and other chemical irritants, hereafter collectively referred to as “toxicants”, in vivo have not been identified, precluding the development of effective countermeasures.

Isocyanates are reactive organic chemicals widely used in the industrial production of polyurethane polymers, pesticides, fungicides and other materials. Methyl isocyanate (MIC), a precursor in pesticide production, was the major causative agent of the environmental disaster in Bhopal, India, responsible for over 3,000 immediate deaths and several thousand additional casualties in the years following the accident (1). In the United States, MIC exposures have occurred following spills of the pesticide metam sodium (sodium N-methyl-dithiocarbamate) in railroad and agricultural accidents (2, 3). In these accidents, metam sodium reacted with soil components and water to produce MIC and other reactive agents (3-5). MIC-exposure caused immediate unbearable irritation of eyes, nose and throat (6). The airways are especially sensitive to MIC and other isocyanates. Dependent on exposure levels and duration, MIC-exposed individuals present with airway hyper responsiveness, inflammation, reactive airway dysfunction syndrome (RADS), and airway edema and injuries (1). Bifunctional isocyanates such as 2,4-toluene-diisocyanate (TDI), diphenylmethane-4,4′-diisocyanate (MDI) and hexamethylene-diisocyanate (HDI), used in the production of polyurethane products, are equally strong irritants and cause asthma-related symptoms upon repeated exposures (7).

The severe irritation following exposures to isocyanates is similar to the incapacitating effects of tear gas agents (8, 9). The development of tear gas agents dates back to World War I, when almost all factions used airway irritants and chemical lacrimatory agents (tear gases), such as acrolein (Papite), chloropicrin (PS), bromoacetone, benzyl bromide and others (9-11). CN tear gas, a riot control agent, was developed in the 1920s and widely used by law enforcement until the 1960s (12). The active lacrimatory agent in CN is 2-chloroacetophenone. Due to its toxicity, CN was supplanted by CS tear gas, containing 2-chlorobenzylidene malononitrile as its active ingredient. CS is currently the most widely used riot control agent world-wide. CR is another modern riot control agent, containing dibenzo[b,f][1,4]oxazepine, as its lacrimatory principle (FIG. 1B) (12).

Despite the infamy of exposures to various chemical irritants/toxicants in occupational and environmental medicine, and the widespread and frequent use of tear gas agents for over 90 years, with possibly millions of exposures, little is known about the molecular and cellular actions of these agents. Current medical treatment for such exposures includes the removal of the toxicants by dilution, washing and chemical neutralization, treatment of pain with anti-inflammatory drugs and general and local anesthetics, and stabilization of the airways with bronchodilators (13). While these procedures are helpful, the additional use of pharmacological agents blocking the specific targets of isocyanates and tear gases would allow a more efficient treatment to alleviate the acute irritation, pain and other noxious effects, and to prevent the development of chronic health effects.

The Transient Receptor Potential A1 (TRPA1) channel (ANKTM1) is a non-selective calcium permeable cation channel, which is also permeable to other cations, such as sodium. Thus, TRPA1 channels modulate membrane potential by modulating the flux of cations such as calcium and sodium ions. Although non-selective cation channels such as TRPA1 modulate, among other things, calcium ion flux, they are mechanistically distinct from voltage-gated calcium channels. Generally, voltage-gated calcium channels respond to depolarization of the potential difference across the membrane and can open to permit an influx of calcium from the extracellular medium and a rapid increase in intracellular calcium levels or concentrations. In contrast, non-selective cation channels are generally signal transduction gated, long lasting, and produce less rapid changes in ion concentration. These mechanistic differences are accompanied by structural differences among voltage-gated and cation permeable channels. Thus, although many diverse channels act to regulate ion flux and membrane potential in various cell types and in response to numerous stimuli, it is important to recognize the significant structural, functional, and mechanistic differences among different classes of ion channels.

While immunological pathways are thought to mediate the allergic sensitization to isocyanates in the airways, studies in animal models point to a role of peripheral sensory C-fibers in their acute noxious effects and in exposure-induced airway hyper reactivity (14-19). In guinea pigs, isocyanates stimulate the release of neuropeptides from capsaicin-sensitive (C-fiber) airway nerve endings, leading to constriction of isolated bronchial segments (20, 21). Similar to the airways, the cornea of the eye is densely innervated by peripheral sensory nerve fibers. A majority of these fibers are trigeminal chemosensory C-fibers that trigger the lacrimation reflex following exposure to a noxious chemical stimulus (22). In addition to lacrimation, activation of corneal C-fibers-induces-ocular pain and blepharospasm, both symptoms associated with tear gas exposures (23). Ocular pre-treatment with local anesthetics abolishes the tear gas-induced lacrimation reflex, suggesting that these agents target corneal chemosensory nerve endings (22).

Peripheral sensory neurons express a large number of excitatory or sensitizing chemosensory receptors, including members of the Transient Receptor Potential (TRP) ion channel family (24, 25, 261). Natural products activating the sensory neuronal TRP channels, TRPV1 and TRPA1, induce effects similar to industrial isocyanates and tear gases. For example, the key ingredient of pepper spray, capsaicin, is a specific agonist of TRPV1 (27, 28). TRPA1 is the receptor for mustard oil (allyl isothiocyanate), the pungent ingredient in mustard, for allicin and diallyl disulfide, the lacrimatory principles in garlic and onions, and pungent natural dialdehyde sesquiterpenes (29-33). In addition to natural products, TRPA1 is also activated by industrial and environmental electrophilic and oxidizing chemicals-(34-36). For example, TRPA1 is activated by hypochlorite, the reactive mediator of the potent irritant gas, chlorine, and is crucial for oxidant-induced respiratory depression and nocifensive behavior in mice (36-38). The role of TRPA1 as a major chemical irritant sensor in airway sensory neurons was shown to be an essential requirement for cigarette smoke extract-induced neurogenic inflammation in mice and guinea pigs, and by findings describing its interaction with endogenous reactive mediators enriched during airway inflammation (39-43).

Recent studies have shown that TRPA1 is activated by chemical tear gas agents in vitro, including acrolein, CN, CS and CR (34, 44). Since these chemicals are highly reactive and may induce non-specific tissue damage, it is questionable whether all of them selectively and potently target TRPA1 in vivo. Reactive agents may be inactivated before reaching sensory neuronal targets, or activate neurons indirectly, through factors released from damaged tissue. For example, adenosine or ATP released from airway tissue damaged by inhalation of organic chemical or acidic fumes have been shown to activate sensory neurons through interaction with purinergic receptors (45). Thus, without detailed whole animal physiological, pharmacological and behavioral studies, it would not be possible to validate TRPA1 as a specific target for any given chemical in vivo.

The molecular targets for industrial isocyanates in sensory neurons are unknown. Isocyanates are highly electrophilic compounds chemically related to isothiocyanates such as mustard oil. Methylisothiocyanate (MITC), the isothiocyanate analog of MIC, is a widely used soil fumigant that frequently causes irritation and occupational injuries in agricultural workers (FIG. 1A) (3, 4). In comparison to mustard oil, MITC is only a weak agonist of TRPA1 in vitro (29). Evidence suggests activation of TRPA1 by reactive chemicals such as isocyanates and isothiocyanates occurs through covalent modification of cytosolic amino acid residues in the N-terminus of the ion channel protein (46, 47). Intriguingly, ruthenium red, a blocker of TRPA1 and other TRP channels, inhibits isocyanate-induced contraction of isolated guinea pig bronchi (21). Thus, activation of sensory neuronal TRP ion channels may contribute to the immediate noxious effects of isocyanate exposures in vivo.

### SUMMARY OF THE INVENTION

The applicants have discovered that TRPA1 is the major mediator of sensory neuronal activation by isocyanates, tear gas agents, vesicants such as sulfur mustard, among other chemical irritants/toxicants, both in vitro and in vivo, and that TRPA1 antagonists selectively block neuronal activation by these agents, providing a basis for a therapy for treating such toxicant exposures.

It is thus an object of the present invention to provide a method of using agents which can modulate TRPA1 function to inhibit the physical effects of chemical irritants/toxicants when given prior to exposure or to lessen the physical effects when administered post exposure, and more specifically, to a method for counteracting the acute physical noxious effects of toxicants, including but not limited to, tear gases, chlorine, hydrogen peroxide, ammonia, phosgene, chloropicrin, isocyanates and mustard gases, including counteracting not only pain, but inflammation, lachrymation, blepharospasm, respiratory irritation and depression, airway mucus secretion, airway obstruction and injury, cough and incapacitation and cutaneous chemical injuries.

In particular, there is provided a method of inhibiting the effects of exposure by a mammal to chemical irritants/toxicants comprising administering an effective amount of a compound that inhibits a TRPA1 function, before or after exposure thereto, wherein the compound blocks the TRPA1 receptor (“TRPA1 inhibitor”) so as to inhibit or counter the physical effects of the chemical irritants/toxicants. Another embodiment also provides a method of preventing or treating a disease or condition in a mammal, which disease or condition includes hypersensitivity to chemical stimuli, particularly in regards to inflammatory airway conditions, such as asthma, rhinitis, etc., comprising administering to the mammal a therapeutically effective amount of a compound that inhibits TRPA1 function, wherein the compound reduces the hypersensitity and mediates the response to such chemical stimuli in the mammal.

Moreover, it is believed that prompt administration could alleviate or reduce the long term effects from an exposure to a toxicant. The long term indications for chemical exposures for TRPA1 activators such as tear gas agents, chlorine, sulfur mustard which may be reduced by practicing the method of the invention include for example, peripheral neuropathy, inducing either numbness or chronic neuropathic pain, reactive airways dysfunction syndrome (RADS), due to lung injury, blindness, due to eye inflammation, skin scarring, hyperpigmentation, folliculitis, pulmonary fibrosis, bronchiectasis, and pneumonia.

The method of the present invention finds particular utility, among numerous others, for inhibiting toxicant effects in emergency, law enforcement and military personnel entering toxicant exposure areas.

### BRIEF DESCRIPTION OF THE DRAWINGS

FIGS. 1A-G show specific industrial isocyanates and tear gases, and that these toxicants activate TRPA1 channels in HEK-293 cells.

FIG. 1A shows chemical structures of three known environmental and occupational irritants, methylisothiocyanate (MITC), methylisocyanate (MIC) and hexamethylene diisocyanate (HDI).

FIG. 1B shows the structures of tear gas agents 2-chloroacetophenone (CN), 2-chlorobenzalmalononitrile (CS), dibenz[b,f][1,4]oxazepine (CR), benzyl bromide and bromoacetone (bromo-2-propanone) and chloropicrin (PS).

FIG. 1C shows the dose-response curves of isocyanate-activated Ca²⁺-influx into hTRPA1-transfected HEK293t cells. The [Ca²⁺]ᵢ induced by each dose is represented as the percentage of maximal [Ca²⁺]ᵢ elicited by a saturating dose of mustard oil (100 μM) applied 75 s later (baseline [Ca²⁺]ᵢ was subtracted). hTRPA1 was activated by MIC (EC₅₀=25±3 μM, n=28±5 cells/dose, red squares) and HDI (EC₅₀=2.6±0.7 μM, n=29±4 cells/dose, black circles).

FIG. 1D shows the dose-response analysis of tear gas agent-activated Ca²⁺-influx into hTRPA1-transfected HEK293t cells. The [Ca²⁺]ᵢ induced by each dose is represented as the percentage of maximal [Ca²⁺]ᵢ elicited by a saturating dose of mustard oil (100 μM) applied 75 s later (baseline [Ca²⁺]ᵢ was subtracted). hTRPA1 was activated by CN (EC₅₀=91±12 nM, n=49±1 cells/dose, green triangles), CS (EC₅₀=7±1 nM, n=52±5, blue squares), CR (EC₅₀=308±150 nM, n=70±9, red circles), PS (EC₅₀=215±62 nM, n=32±4, grey diamonds), benzyl bromide (BenzBr, EC₅₀=12±1 μM, n=66±17, black triangles) and bromoacetone (BrAc, EC₅₀=1.1±0.3 μM, n=78±14 cells/dose, purple stars).

FIG. 1E shows that the application of MIC (100 μM, purple bar) induces an increase of single channel openings of an excised patch in the inside-out configuration from hTRPA1 over-expressing CHO cells. The voltage was held at −40 mV, bath solution contained 0.5 mM PPPᵢ and devoid of Ca²⁺ (10 mM EGTA).

FIG. 1F shows that the application of CS (10□M, blue bar) induces an increase of single channel openings in an excised patch in the inside-out configuration from hTRPA1 over-expressing CHO cells. The conditions were the same as FIG. 1E.

FIG. 1G shows the responses of hTRPA1 mutant-expressing HEK-293t cells to 100 μM MIC, 100 μM HDI, 100 μM CS, 100 μM CN, 300 μM CR, 100 μM bromoacetone (BrAc) and 100 μM benzyl bromide (BenzBr). Increase in [Ca²⁺]ᵢ is displayed as percentage of [Ca²⁺]ᵢ activated by a saturating dose of carvacrol (300 μM). Mutants tested are coded as follows: K (grey)=K708 (MIC n=84, HDI n=53, CS n=157, CN n=108, CR n=154, BrAc n=44, BenzBr n=64 cells), 3C (white)=C619, C639, and C663 combined (MIC n=34, HDI n=47, CS n=96, CN n=56, CR n=90, BrAc n=25, BenzBr n=40 cells), 3CK (black)=C619, C639, C663, and K708 combined (MIC n=30, HDI n=123, CS n=129, CN n=18, CR n=17, BrAc n=38, BenzBr n=41).

FIG. 2A-F illustrate that the industrial isocyanates and tear gas agents activate native TRPA1 channels in cultured sensory neurons.

FIG. 2A shows the industrial isocyanates induced Ca²⁺-influx into cultured mouse DRG neurons, as measured by fluorescent Fura-2 imaging. Neurons are shown before activation (Pre, left column), 70 s after challenge (middle column) with MIC (100 μM, top row) or HDI (100 μM, bottom row) and following application of 5 μM capsaicin (Cap, right column) after 50 s. Pseudocolors denote 0-3 μM [Ca²⁺]ᵢ. Original magnification, ×10.

FIG. 2B shows the average [Ca²⁺]ᵢ of mouse DRG neurons (thick lines) with an application of MIC (100 μM, n=168 neurons from 2 mice, black line) or HDI (100 μM, n=270 from 2 mice, red line), followed by 100 μM mustard oil, 5 μM capsaicin (Cap) and 65 mM KCl. The thin lines represent SEM.

FIG. 2C shows the tear gas agent-induced Ca²⁺ influx into cultured murine DRG neurons, as measured by fluorescent Fura-2 imaging. Neurons are shown before activation (Pre, left column), 70 s after challenge (middle column) with CS (100 μM, top row), CN (100 μM, middle row) or CR (300 μM, bottom row) and following application of 5 μM capsaicin (Cap, right column) after 50 s. Pseudocolors denote 0-3 μM [Ca²⁺]ᵢ. Original magnification, ×10.

FIG. 2D shows the average [Ca²⁺]ᵢ of mouse DRG neurons (thick lines) with an application of CS (100 μM, blue line, n=161 neurons from 2 mice), CN (100 μM, green line, n=335 from 5 mice) or CR (300 μM, red line, n=137 from 2 mice), followed by 100 μM mustard oil, 5 μM capsaicin (Cap) and 65 mM KCl. Thin lines represent SEM.

FIG. 2E shows the dose-response curves of isocyanate-activated Ca²⁺-influx into mouse DRG neurons are similar to hTRPA1-transfected HEK293t cells. The [Ca²⁺]ᵢ induced by each dose is represented as the percentage of maximal [Ca²⁺]ᵢ elicited by a saturating dose of mustard oil (AIC, 100 μM) applied 75 s later (baseline [Ca²⁺]ᵢ was subtracted). AIC-sensitive mouse DRG neurons were activated by MIC (EC₅₀=36±7 n=30±6 neurons/dose, solid black squares) and HDI (EC₅₀=8.4±1.4 μM, n=39±12/dose, solid red squares). Dashed lines and open circles represent hTRPA1-transfected HEK293t cells as shown in FIG. 1C.

FIG. 2F shows the dose-response curves of tear gas agent-activated Ca²⁺-influx into mouse DRG neurons are right-shifted compared to responses in hTRPA1-transfected HEK293t cells. The [Ca²⁺]ᵢ induced by each dose is represented as the percentage of maximal [Ca²⁺]ᵢ elicited by a saturating dose of mustard oil (AIC, 100 μM) applied 75 s later (baseline [Ca²⁺], was subtracted). AIC-sensitive mouse DRG neurons were activated by CS (EC₅₀=12.1±0.3 μM, n=41±9 neurons/dose, solid blue squares), CN (EC₅₀=6±1 μM, n=23±5/dose, solid green squares) and CR (EC₅₀=246±27 μM, n=37±16/dose, solid red squares). Dashed lines with open circles represent dose response curves of Ca²⁺-influx into hTRPA1-transfected HEK293t shown in FIG. 1D.

FIG. 3A-F show that CN induces TRPA1-like currents in mouse DRG neurons.

FIG. 3A shows the TRPA1-like current-voltage curves of a representative mouse DRG neuron before activation (black trace), activation by 100 μM CN (green trace) and inhibition by ruthenium red (10 μM, red trace) in the whole-cell configuration. V_{holding}=0 mV to minimize voltage-gated channels. Currents were measured with a voltage ramp from −100 mV to +100 mV over 100 ms at 0.5 Hz intervals. Intracellular Cs-based solution with 10 mM EGTA was used.

FIG. 3B shows the average native TRPA1-like currents at −80 mV and +80 mV in mouse DRG neurons superfused with 100 μM CN (black bar), followed by ruthenium red (10 μM) as described in FIG. 1b (n=4 out of 16 neurons). Baseline current was subtracted for each trace.

FIG. 3C shows the hTRPA1 current-voltage curves before activation (black trace), at maximal activation by 10 μM CN (green trace) and after inactivation phase (blue trace) in the whole-cell configuration. Currents were measured with a voltage ramp from −80 mV to +80 mV over 100 ms at 0.5 Hz intervals, V_{holding}=0 mV. Intracellular Cs-based solution with 10 mM EGTA was used.

FIG. 3D shows the averaged TRPA1 currents at −80 mV and +80 mV in hTRPA1-transfected HEK-293t cells superfused with 10 μM CN (black bar) as described in FIG. 1C (n=4).

FIG. 3E shows the current-voltage relationship of HDI (100 μM)-activated hTRPA1 single channel currents, recorded in the cell-attached configuration from hTRPA1-expressing CHO cells. In the absence of extracellular Ca²⁺ in the pipette (red line) the I-V relationship is linear (averaged over 3 patches). In the presence of 2 mM Ca²⁺ in the pipette, single channel conductance is reduced and the I-V relationship is outwardly rectifying (green line) (n=3 patches).

FIG. 3F shows representative hTRPA1 single channel openings activated by HDI (100 μM) at +60, 0 and −60 mV, recorded in the cell attached configuration, in the absence (left, showing two channels) and presence (right, showing three channels) of Ca²⁺ (2 mM). Single channel conductance is visibly reduced in the presence of Ca²⁺.

FIG. 4A-F show ablation of isocyanate and tear gas agent induced sensory neuronal activation by genetic ablation or pharmacological blockade of TRPA1.

FIG. 4A shows that isocyanate-induced Ca²⁺ influx is absent in DRG neurons from Trpa1^{-/-} mice. Neurons are shown before application (Pre, left column), 70 s after challenge (middle column) with MIC (100 μM, top row) or HDI (100 μM, bottom row), and following 5 μM capsaicin (Cap, right column) after 50 s. Pseudocolors denote 0-3 μM [Ca²⁺]ᵢ. Original magnification, ×10.

FIG. 4B shows the average [Ca²⁺]ᵢ of mouse DRG neurons (thick lines) with an application of MIC (100 μM, red line, n=217 from 2 mice), HDI (100 μM, black line, n=204 neurons from 2 mice), followed by 100 μM mustard oil, 5 μM capsaicin (Cap) and 65 mM KCl. The thin lines represent SEM.

FIG. 4C shows that tear gas agent induced Ca²⁺ influx is absent in DRG neurons from Trpa1^{-/-} mice, shown before activation (Pre, left column), 70 s after challenge (middle column) with CS (100 μM, top row), CN (100 μM, middle row) or CR (300 μM, bottom row), and following by 5 μM capsaicin (Cap, right column) after 50 s. Pseudocolors denote 0-3 [Ca²⁺]ᵢ. Original magnification, ×10.

FIG. 4D shows the average [Ca²⁺]ᵢ of mouse DRG neurons (thick lines) with an application of CS (10 μM, blue line, n=229 neurons from 2 mice), CN (100 μM, green line, n=270 neurons from 5 mice) or CR (300 μM, red line, n=108 neurons), followed by 100 μM mustard oil, 5 μM capsaicin (Cap) and 65 mM KCl. The hin lines represent SEM.

FIG. 4E shows the dose-response curves of inhibition of industrial isocyanate or tear gas agent-activated Ca²⁺-influx into mouse DRG neurons by the TRPA1-antagonist HC-030031. The [Ca²⁺]ᵢ induced by each dose is represented as the percentage of [Ca²⁺]ᵢ elicited by a saturating dose of capsaicin (5 μM, Cap) applied 125 s later (baseline [Ca²⁺]ᵢ was subtracted). HC-030031 inhibited the [Ca²⁺]ᵢ induced by 10 μM HDI (IC₅₀=74±3 μM, n=31±4 Cap-sensitive neurons/dose, black triangles) 10 μM CS (IC₅₀=4.5±0.4 μM, n=26±6/dose, blue squares) and 10 μM CN (IC₅₀=884±23 nM, n=25±5/dose, green circles) in mouse DRG neurons.

FIG. 5A-D show ablation of isocyanate and tear gas agent-induced nocifensive responses in mice by genetic deletion or pharmacological blockade of TRPA1.

FIG. 5A shows the nocifensive responses following application of 10 μl of 100 mM HDI (n=6) or 100 mM CS (n=6) or 100 mM CN (n=9) to the eye of untreated C57/B16 wild-type mice (black bars), and the same mice following an injection of 1 mg, i.p. (50 mg/kg, grey bar) or 6 mg, i.p. (300 mg/kg, white bar) of the TRPA1-antagonist HC-030031. Nocifensive responses were quantified by counting strokes of the orbitofacial area on the observation chamber floor over 2 min for CS and CN for the 300 mg/kg experiments and over 3 min for the other treatments. ** indicates significance (p<0.01), * (p<0.05).

FIG. 5B shows the nocifensive responses (licks, lifts and flicks) over 3 min following 25 μl subplantar injections of 4 mM CN or 6 mM HDI (n=6/group) into the hind paw of untreated C57/B16 wild-type mice (black bars), and the same mice following an injection of 2 mg, i.p. (100 mg/kg, grey bars). ** indicates significance (p<0.01), * (p<0.05).

FIG. 5C shows a comparison of nocifensive responses of wild-type (black bars) and Trpa1−/− mice (white) following application of 10 μl of 200 mM HDI, 100 mM CS or 100 mM CN to the right eye. Strokes of the orbitofacial area against the observation chamber floor were counted over 2 min for CS and CN, and for 3 min for HDI (n=6 wild-type and n=6 Trpa1−/− mice were tested with CN, n=6 wild-type and n=7 Trpa1−/− mice tested with CS, and n=6 wild-type and n=7 Trpa1−/− mice were tested with HDI). *** indicates significance (p<0.001), ** (p<0.01) and * (p<0.05).

FIG. 5D shows the nocifensive responses (licks, lifts and flicks) over 5 min following 25 μl subplantar injections of 2 mM CN (n=8/group) or 4 mM bromoacetone (BrAc, n=6/group) into the hind paw of Trpa1^{+/+} and Trpa1^{-/-} mice. * indicates significance (p<0.05).

FIG. 6A-F show the effects of isocyanates and tear gas agents on [Ca²⁺]ᵢ in mock-transfected (pcDNA3) or rTRPV1-transfected HEK-293t cells, and in mouse sensory neurons with or without a PLC-inhibitor.

FIG. 6A shows an average ratiometric fura-2 fluorescence emission of mock-transfected (pcDNA3) HEK-293t cells (thick lines) during application of MIC (100 μM, red line), HDI (100 μM, black line), CN (100 μM, green line), CS (100 μM, blue line), CR (300 μM, yellow line), benzyl bromide (BenzBr, 100 μM, orange line) or bromoacetone (BrAc, 100 μM, purple line), followed by 5 μM ionomycin (50 cells/experiment). The thin lines represent SEM.

FIG. 6B shows an average industrial isocyanate or tear gas agent-activated Ca²⁺-influx into rTRPV1-transfected HEK293t cells. The average [Ca²⁺]ᵢ (thick lines) for each toxicant is represented as the percentage of maximal [Ca²⁺]ᵢ elicited by a saturating dose of capsaicin (5 μM) applied 75 s later (baseline [Ca²⁺]ᵢ was subtracted). rTRPV1 was not activated by MIC (100 μM, red line, n=19), HDI (100 μM, black line, n=37), CN (100 μM, green line, n=86), CS (100 μM, blue line, n=60), CR (300 μM, yellow line, n=59), benzyl bromide (BenzBr, 100 μM, orange line, n=32) or bromoacetone (BrAc, 100 μM, purple line, n=57). The thin lines represent SEM.

FIG. 6C shows an average ratiometric fluorescence emission of fura-2 of mouse DRG neurons (thick lines) with an application of 100 μM bromoacetone (BrAc, orange line, n=71 neurons) or 100 μM benzyl bromide (BenzBr, purple line, n=40 neurons) followed by 100 μM mustard oil, 5 μM capsaicin (Cap) and 65 mM KCl. The thin lines represent SEM.

FIG. 6D shows an average ratiometric fluorescence emission of fura-2 of mouse DRG neurons (thick lines) incubated in the presence of a Phospholipase C-Inhibitor (4 μM, ET-18-OCH₃). Application of MIC (100 μM, red line, n=56), HDI (100 μM, black line, n=83), CN (100 μM, green line, n=121), CS (100 μM, blue line, n=92) and CR (500 μM, purple line, n=55), followed by 100 μM mustard oil, 5 μM capsaicin (Cap) and 65 mM KCl were similar to results without the ET-18-OCH₃. The thin lines represent SEM.

FIG. 6E shows an average [Ca²⁺]ᵢ of mouse trigeminal ganglia neurons (thick lines) with an application of HDI (100 μM, black line, n=139 neurons), CS (100 μM, blue line, n=27) or CN (100 μM, green line, n=31), followed by 100 μM mustard oil, 5 μM capsaicin (Cap) and 65 mM KCl.

FIG. 6F shows a dose-response analysis of CN tear gas agent-activated Ca²⁺-influx into mTRPA1- and hTRPA1-transfected HEK293t cells and mouse DRG neurons. The [Ca²⁺]ᵢ induced by each dose is represented as the percentage of maximal [Ca²⁺]ᵢ elicited by a saturating dose of mustard oil (100 μM) applied 75 s later (baseline [Ca²⁺]ᵢ was subtracted). CN activated AIC-sensitive mouse DRG neurons (EC₅₀=6±1 μM, n=23±5/dose, red circles), hTRPA1 (EC₅₀=91±12 nM, n=49±1 cells/dose, black squares) and mTRPA1 (EC₅₀=66±14 nM, n=84±20, blue triangles).

FIG. 7a-E show the activation of single channel openings of hTRPA1 channels in CHO cells by isocyanates and tear gas agents.

FIG. 7A shows a current amplitude histograms represent the occurrence of distinct current amplitudes during a representative 10 s recording from an inside out patch of hTRPA1 expressing CHO cells before (right panel) and after application of 100 μM MIC (left panel). Voltage was held at −40 mV, bath solution contained 0.5 mM PPPi, 10 mM EGTA.

FIG. 7B shows a current amplitude histograms represent the occurrence of distinct current amplitudes during a representative 10 s recording from an inside-out patch of a hTRPA1 over-expressing CHO cell before (right panel) and after application of 10 μM CS (left panel). Voltage-potential was held at −40 mV, bath solution contained 0.5 mM PPPi, 10 mM EGTA.

FIG. 7C shows representative single channel currents before (left panel) and after application of 100 mM HDI (right panel) at +60, +40, +20, 0, −20, −40 and −60 mV, recorded in the cell-attached configuration from a hTRPA1-expressing CHO cell in the presence of 10 mM EGTA and without Ca²⁺.

FIG. 7D shows representative hTRPA1 single channel openings activated by HDI (100 μM) at +60, 0 and −60 mV, recorded in the cell attached configuration as described in FIG. 7C, in the absence (left, showing two channels) and presence (right, showing three channels) of Ca²⁺ (2 mM). Single channel conductance is visibly reduced in the presence of Ca²⁺.

FIG. 7E shows a current-voltage relationship of HDI (100 μM)-activated hTRPA1 single channel currents, recorded in the cell-attached configuration from hTRPA1-expressing CHO cells. In the absence of extracellular Ca²⁺ in the pipette (red line) the I-V relationship is linear (averaged over 3 patches). In the presence of 2 mM Ca²⁺ in the pipette, single channel conductance is reduced and the I-V relationship is outwardly rectifying (green line) (n=3 patches).

FIG. 8A-C show a block of tear gas agent-induced TRPA1 activity by pharmacological antagonists.

FIG. 8A shows that a CS (10 nM, top row), CN (100 nM, middle row) or CR (1 μM, bottom row)-induced Ca²⁺ influx in hTRPA1-transfected HEK-293t (right column) is blocked in the presence of TRPA1-antagonists AP-18 (25 μM, middle column) or HC-030031 (25 μM, right column). [Ca²⁺]ᵢ was measured by Fura-2 imaging. Images were taken 120 seconds following application of tear gas agent. Pseudocolors denote 0-3 μM [Ca²⁺]ᵢ. Original magnification, ×20.

FIG. 8B shows CS (10 μM, top row), CN (10 μM, middle row) or HDI (10 μM, bottom row)-induces a Ca²⁺ influx into cultured mouse DRG neurons (left column) after 75 s, this effect is absent in DRG neurons incubated for 5-30 min with the TRPA1-antagonist HC-030031 (100 μM, middle column). The TRPA1-antagonist treated DRG neurons are responsive to the TRPV1-agonist capsaicin (5 μM, Cap, right column). Pseudocolors denote 0-3 [Ca²⁺]ᵢ. [Ca²⁺]ᵢ was measured by Fura-2 imaging. Original magnification, ×10.

FIG. 8C shows that an increase of [Ca²⁺]ᵢ in DRG neurons (black line) activated by 10 μM CS, followed by 100 μM mustard oil (MO), 5 μM capsaicin (Cap) and 65 mM KCl. CS-induced neuronal Ca²⁺-influx is blocked in the presence of TRPA1 antagonists HC-030031 (25 μM, green) or AP-18 (25 μM, purple). Responses of n=108±23 neurons were averaged per dose. The thin lines represent SEM.

FIGS. 9A and B show that the genetic ablation or pharmacological block of TRPA1 inhibits vesicant-induced edema in the mouse ear.

FIG. 9A shows mouse ear thickness was measured as a sign of edema with a digital caliper 24 h after the application of 500 ng of the skin vesicant CEES (2-chloroethyl-ethyl-sulfide) onto the skin (in CH₂Cl₂) and compared to a contralateral control ear.

FIG. 9B shows discs of mouse ears treated with 500 ng CEES were punched 24 h after the application of CEES, weighed and compared to discs from the contralateral control ear. All values are represented as % increase compared to contralateral control ear. Mice in experimental conditions MC and HC received the treatment of vehicle and TRPA1−/− antagonist 1 h post-exposure to CEES. (WT wild type, TRPA1−/− TRPA1 deficient mice, MC mice treated with methylcellulose vehicle, HC mice treated with TRPA1 antagonist HC-030031, 200 mg/kg 1 h, 8 h and 16 h after CEES exposure) Asterisks indicate significance (*p<0.05, **p<0.01; Student's T-Test). TRPA1−/− mice, and mice treated with HC-030031 show dramatically diminished ear edema.

FIGS. 10A-F compare the histological sections of mice ears treated with CEES compared to sections from the contralateral control ear. FIGS. 10A, B and C show 10 μM thick sections of contralateral control ears, FIGS. 10 D, E, and F show sections of ears treated with 500 ng CEES. The A and D sections were from wild-type (WT) mice; The B and E sections were from WT mice treated with TRPA1-antagonist HC-030031 (200 mg/kg 1 h prior, and 8 h and 16 h post-treatment with CEES); The C and F sections were of TRPA1-deficient mice (TRPA1−/−). All pictures were taken at 100× magnification. Mice in B and E received treatment with HC-030031 30 min prior the application of CEES. The scale bar in C represents 100 μm.

### DETAILED DESCRIPTION OF THE PREFERRED EMBODIMENT

The following terms, among others, are used to describe the present invention. It is to be understood that a term which is not specifically defined is to be give a meaning consistent with the use of that term within the context of the present invention as understood by those of ordinary skill.

The term “compound”, as used herein, unless otherwise indicated, refers to any specific chemical compound disclosed herein and includes tautomers, regioisomers, geometric isomers, and where applicable, optical isomers (enantiomers) thereof, as well as pharmaceutically acceptable salts and derivatives (including prodrug forms) thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity.

The term “patient” or “subject” is used throughout the specification within context to describe an animal, generally a mammal and preferably a human, to whom treatment, including prophylactic treatment (prophylaxis), with the compositions according to the present invention is provided. For treatment of those infections, conditions or disease states which are specific for a specific animal such as a human patient, the term patient refers to that specific animal. In certain important aspects of the present invention, TRPA1 antagonists find use to inhibit the effects of toxicants and provide protection for emergency, law envorcement and military personnel entering toxicant exposure areas.

The term “effective” is used herein, unless otherwise indicated, to describe an amount of a compound or composition which, in context, is used to produce or effect an intended result, whether that result relates to the inhibition of the effects of a toxicant on a subject or the treatment of a subject for secondary conditions, disease states or manifestations of exposure to toxicants as otherwise described herein. This term subsumes all other effective amount or effective concentration terms (including the term “therapeutically effective” which are otherwise described in the present application.

The terms “treat”, “treating”, and “treatment”, etc., as used herein, refer to any action providing a benefit to a patient at risk for or afflicted by exposure to a toxicant or chemical irritant, including improvement in the condition through lessening or suppression of at least one symptom, delay in progression of the noxious effects of the exposure, prevention or delay in the onset of noxious effects of the exposure, etc. Treatment, as used herein, encompasses both prophylactic and therapeutic treatment.

The terms “toxicant”, “irritant” and/or “irritant/toxicant” are used synonymously to describe chemical agents, primarily, but not exclusively gaseous compounds, which activate TRPA1, resulting in manifestations, disease states and conditions including pain and conditions, effects and disease states which are otherwise described herein. These chemical agents include industrial irritants and chemical weapons such as chlorine, hydrogen peroxide, ammonia, phosgene, chloropicrin, isocyanates (hexamethylenediisocynate, methylisocyanate), among others, including tear gases and mustard gases (sulfur and nitrogen).

The term “mustard gas” of which “sulfur mustard” and “nitrogen mustard” are subclasses is used to describe a class of related cytotoxic, vesicant chemical warfare agents with the ability to form large blisters on exposed skin. Pure sulfur mustards are colorless, viscous liquids at room temperature. However, when used in impure form as warfare agents they are usually yellow-brown in color and have an odor resembling mustard plants, garlic or horseradish, hence the innocuous name. Mustard agents are regulated under the 1993 Chemical Weapons Convention (CWC). Three classes of chemicals are monitored under this Convention, with sulfur and nitrogen mustard grouped in Schedule 1, as substances with no use other than chemical warfare.

A list of effective sulfur mustard agents commonly stock-piled is as follows:
1,2-Bis(2-chloroethylthio) ethane (aka Sesquimustard; Q)
1,3-Bis(2-chloro ethylthio)-n-propane
1,4-Bis(2-chloroethylthio)-n-butane
1,5-Bis(2-chloroethylthio)-n-pentane
2-Chloroethylchloromethylsulfide
Bis(2-chloroethyl) sulfide (HD)
Bis(2-chloroethylthio) methane
Bis(2-chloroethylthiomethyl)ether
Bis(2-chloroethylthioethyl)ether (O Mustard)

Examples of nitrogen mustards that may be used for chemical warfare purposes and their military weapon designations include:
HN1: Bis(2-chloroethyl)ethylamine
HN2: Bis(2-chloroethyl)methylamine
HN3: Tris(2-chloroethyl) amine

The term “tear gas” or “lachrymator” is used to describe a lachrymatory agent which is a chemical compound that stimulates the corneal nerves in the eyes to cause tearing, pain, and even temporary blindness. Common lachrymators include CS (2-chlorobenzylidene malononitrile), CR (dibenz[b,f][1,4]oxazepine, CN (2-chloroacetophenone), bromoacetone, phenacyl bromide, benzyl bromide, bromoacetone and xylyl bromide, among others, as otherwise described herein. Lacrymators often share the chemical structural element Z═C—C—X, where Z=carbon or oxygen, and X=bromide or chloride.

Tear gases or lachrymatory agents are commonly used as riot control agents and chemical warfare agents. For example, tear gas and pepper spray are commonly used for riot control.

The term “acyl” is art-recognized and refers to a group represented by the general formula hydrocarbylC(O)—, preferably alkylC(O)—.

The terms “acylamino” is art-recognized and refers to a moiety having an amino group and an acyl group and may include substitutents on same as otherwise disclosed herein.

The term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The terms “alkoxyl” or “alkoxy” as used herein refers to an alkyl group, as defined below, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m is 0 to 10 and R₈ is an aryl or substituted aryl group, a cycloalkyl group, a cycloalkenyl, a heterocycle or a polycycle (two or three ringed).

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer, and most preferably 10 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m is 0 or an integer from 1 to 8 and R₈ is the same as defined below (for amine/amino). Representative alkylthio groups include methylthio, ethylthio, and the like.

The terms “amine” and “amino” are art-recognized and refer to both unsubstituted and substituted amines, e.g., a moiety that can be represented by the general formula:

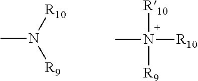

wherein R₉, R₁₀ and R′₁₀ each independently represent a hydrogen, an alkyl, an alkenyl, —(CH₂)ₐ, —R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In certain such embodiments, neither R₉ and R₁₀ is attached to N by a carbonyl, e.g., the amine is not an amide or imide, and the amine is preferably basic, e.g., its conjugate acid has a pKₐ above 7. In even more preferred embodiments, R₉ and R₁₀ (and optionally, R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “amido” is art-recognized as an amino-substituted carbonyl and includes a moiety that can be represented by the general formula:

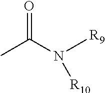

wherein R₉, R₁₀ are as defined above. Preferred embodiments of the amide will not include imides that may be unstable.

The term “aralkyl”, as used herein, refers to an alkyl group substituted with an aryl group (e.g., an aromatic or heteroaromatic group).

The term “aryl” as used herein includes 5-, 6-, and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, polycyclyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The term “carbocycle”, as used herein, refers to an aromatic or non-aromatic ring in which each atom of the ring is carbon.

The term “carbonyl” is art-recognized and includes such moieties as can be represented by the general formula:

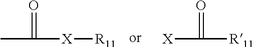

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or—(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The term “electron withdrawing group” refers to chemical groups which withdraw electron density from the atom or group of atoms to which electron withdrawing group is attached. The withdrawal of electron density includes withdrawal both by inductive and by delocalization/resonance effects. Examples of electron withdrawing groups attached to aromatic rings include perhaloalkyl groups, such as trifluoromethyl, halogens, azides, carbonyl containing groups such as acyl groups, cyano groups, and imine containing groups.

The term “ester”, as used herein, refers to a group —C(O)OR₉ wherein R₉ represents a hydrocarbyl group.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures (which can be cyclic, bicyclic or a fused ring system), preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “sulfamoyl” is art-recognized and includes a moiety represented by the general formula:

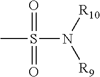

Where R₉ and R₁₀ are as described above.

The term “sulfate” is art-recognized and includes a moiety represented by the general formula:

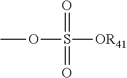

Where R₄₁ is an electron pair, hydrogen, alkyl, cycloalkyl or aryl.

The term “sulfonamido” is art-recognized and includes a moiety represented by the general formula:

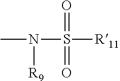

Where R₉ and R′₁₁ are as described above.

The term “sulfonate” is art-recognized and includes a moiety represented by the general formula:

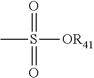

Where R₄₁ is an electron pair, hydrogen, alkyl, cycloalkyl or aryl.

The term “sulfoxido” or “sulfinyl” is art-recognized and includes a moiety represented by the general formula:

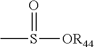

Where R₄₄ is selected from the group consisting of hydrogen, alkyl, alkenyl, alkynyl, cycloalkyl, heterocyclyl, aralkyl or aryl.

The term “thioester” is art-recognized and is used to describe a group —C(O)SR⁹ or —SC(O)R⁹ wherein R9 represents a hydrocarbyl group.

As used herein, the definition of each expression of alkyl, m, n, etc. when it occurs more than once in any structure, is intended to reflect the independence of the definition of the same expression in the structure.

The term “TRPA1”, “TRPA1 protein” and “TRPA1 channel” are used interchangeably to refer to the TRPA1 ion channel.

The terms “antagonist” and “inhibitor” are used interchangeably to refer to an agent, especially including chemical agents which are specifically disclosed herein that decreases or suppresses a biological activity, such as to repress an activity of an ion channel, and in particular a TRPA1 ion channel.

It has been determined that the ion channel TRPA1 is the sensory neuronal receptor for various chemical irritants/toxicants, such as the tear gas agents CN (2-chloroacetophenone), CS (2-chlorobenzylidene malononitrile), CR (dibenz[b,f][1,4]oxazepine), benzylbromide, phenacyl bromide and bromoacetone, in pain-sensing peripheral sensory neurons. In particular, TRPA1 has been determined to be the receptor for industrial and related irritants, including chlorine, hydrogen peroxide, ammonia, phosgene, chloropicrin and isocyanates (hexamethylenediisocynate, methylisocyanate), among others. Moreover, tear gases, sulfur mustard gases, chlorine and hydrogen peroxide have been shown to activate TRPA1 to induce pain behavior and respiratory depression in mice in vivo.

It has been determined that various TRPA1 channel antagonists, which may include but are not limited to, ruthenium red, HC-030031 and AP-18 (formula information below), block activation of TRPA1 by various toxicants which include, but are not limited to chlorine, hydrogen peroxide, ammonia, tear gas agents, chloropicrin and phosgene, as proven in cultured sensory neurons and heterologous cells. HC-030031 in particular blocked the noxious effects of CN, CS, ammonia, bromoacetone and isocyanates in vivo. Thus TRPA1 antagonists find use to counteract the noxious effects of tear gas agents, chlorine, hydrogen peroxide, ammonia, phosgene, chloropicrin and industrial isocyanates. This has been confirmed by tests showing that TRPA1-deficient mice are insensitive to the noxious physical effects of tear gases and lack chlorine and hydrogen peroxide induced respiratory depression.

TRPA1 was thus determined to be a crucial mediator of vesicant-induced injury. Vesicants such as sulfur mustard (Bis(2-chloroethyl) sulfide), the active constituent of mustard gas, induce chemical burns, skin edema, blistering, apoptosis and inflammation. It was discovered that TRPA1-deficient mice were protected from such vesicant injury, tested in the mouse ear vesicant model. This mode is generated by application of a sulfur mustard analog, CEES (2-chloroethyl ethyl sulfide), to the mouse ear. TRPA1-deficient mice showed diminished ear swelling, ear punch weight and diminished edema, measured through pathological analysis. Treatment of mice with a TRPA1 antagonist (HC-030031), pre- and post-exposure to CEES effectively reduced ear swelling, punch weight and edema, establishing that TRPA1 antagonists protect from vesicant-induced injury when administered either before or after contact with the vesicant.

Quite surprisingly, TRPA1 channel antagonists can be used in a method for counteracting the acute physical noxious effects of tear gases, chlorine, hydrogen peroxide, ammonia, phosgene, chloropicrin, isocyanates, sulfur mustard gases pre- and post-exposure, including counteracting not only pain, but inflammation, lachrymation, blepharospasm, respiratory irritation and depression, airway mucus secretion, airway obstruction and injury, cough and incapacitation and cutaneous chemical injuries.

Thus, the method of the invention, by administering antagonists of TRPA1, can effectively inhibit injury caused by vesicants such as sulfur mustard, when administered pre- and post-exposure, as well as for the treatment of cutaneous-chemical-injury and inflammation.

In addition, whether pre or post exposure, administering TRPA1 antagonists may be effective to prevent or reduce the hypersensitivity responses to chemical stimuli in patients affected by inflammatory conditions in the airways or skin, including asthma, rhinitis, chronic obstructive pulmonary disease (COPD), inflammatory skin conditions and others. Asthma and rhinitis patients routinely display heightened sensitivity to chlorine, ammonia and hydrogen peroxide (in household bleach and cleaners), and are at high-risk for injury and incapacitation during tear gas exposures. Thus, as TRPA1 is the major mediator of sensory neuronal activation by such toxicants, TRPA1 antagonists may be used to selectively block neuronal activation by these agents, providing a prophylactic as well as a therapeutic agent for inhibiting the noxious physical effects normally exhibited from such chemical exposures.

### EXAMPLES

### Materials and Methods

Animals

Mice were housed at an AAALAC accredited facility in standard environmental conditions (12 hr light-dark cycle and ˜23° C.). All animal procedures were approved by the Yale Institutional Animal Care and Use Committee. Animals were identically matched for age (12-22 weeks) and gender and the experimenter was blind to the genotype. Trpa1−/− mice were a gift from David Julius (UCSF) and were genotyped as described (33). C-57 mice were purchased commercially (Charles River Laboratories, Mass., USA). In certain experiments, 200 μl intraperitoneal injections of 0, 1, 2 or 6 mg HC-030031 dissolved in 0.5% methylcellulose (Methocel, Fluka, Switzerland) Were administered to mice.

Cell Culture

Adult mouse dorsal root ganglia and trigeminal ganglia were dissected and dissociated by 1 hr incubation in 0.28 WU/mL Liberase Blendzyme 1 (Roche, Germany), followed by washes with Hank's buffered saline, trituration, and straining (70 μM, Falcon, Mass., USA). Trigeminal ganglia were further purified using centrifugation over a Percoll gradient (GE Healthcare, UK). Neurons were cultured in Neurobasal-A medium (Invitrogen) with B-27 supplement, 0.5 mM glutamine and 50 ng/mL NGF (Calbiochem, Merck, Darmstadt, Germany) on 8-well chambered coverglass or 35 mm dishes (Nunc, Denmark) coated with polylysine (Sigma) and laminin (Invitrogen). HEK-293t and CHO cells for Ca²⁺ imaging and electrophysiology were cultured and transfected with human and mouse TRPA1, mutant TRPA1, rat TRPV1 or empty vector (pcDNA3) cDNAs as described (29, 33).

Chemicals and Solutions

If not otherwise indicated, chemicals were purchased from Sigma (St. Louis, Mo., USA). Whole-cell electrophysiological and Ca²⁺-imaging experiments were performed in modified standard Ringer's bath solution (in mM): 140 NaCl, 5 KCl, 2 CaCl₂, 2 MgCl₂, 10 HEPES-NaOH, 5 Glucose, pH 7.3, 315-320 mOsm. Pipette and chip solutions for whole-cell intracellular application contained (in mM): 75 CsCl, 70 CsF, 2 MgCl₂, 10 EGTA, 10 HEPES-CsOH, pH 7.3, 315-320 mOsm. Pipette and bath solutions for single-channel electrophysiological recordings contained identical solutions to the standard Ringer's bath solution with the exception of being Ca²⁺-free and containing 10 mM EGTA. Solutions for recordings in the inside-out configuration contained 0.5 mM sodium tripolyphoshate (PPPi, Acros Organics, NJ, USA). In certain cell-attached recordings, solutions contained 2 mM CaCl₂ and did not contain EGTA and PPPi. Isocyanate solutions of methylisocyanate (MDI, Chem Service Inc., West Chester, Pa., USA) and hexamethylenediisocyanate (HDI), and tear gas solutions of 2-chloroacetophenone (CN), 2-chlorobenzylidene malononitrile (CS, Scientific Exchange, Inc., Center Ossipee, N.H., USA)) and dibenzo[b,f][1,4]oxazepine (CR, Key Organics Ltd, Camelford, UK) were initially dissolved in DMSO at 40 mM. Ionomycin (4 mM, MP Biomedicals, Solon, Ohio), capsaicin (100 mM) and 1-O-octadecyl-2-O-methyl-sn-glycero-3-phosphoryl-choline (20 mM, ET-18-OCH3) were dissolved in ethanol and ruthenium red (100 mM, Latoxan, Valence, France) was dissolved in water. Stock solutions were diluted to their final concentration in appropriate solution for applications. For eye applications, HDI, CN and CS were dissolved in 75% DMSO/PBS to 100 mM. A freezing point osmometer (Advanced Instruments, Norwood, Mass., USA) was used to measure the osmolarity of all solutions. The TRPA1-antagonists 4-(4-Chlorophenyl)-3-methylbut-3-en-2-oxime (10 mM, AP-18, Maybridge, Trevillett, UK) and 2-(1,3-dimethyl-2,6-dioxo-1,2,3,6-tetrahydro-7H-purin-7-yl)-N-(4-isopropylphenyl)acetamide (20 mM, HC-030031, Hydra Bioscience, Cambridge, Mass.) were dissolved in DMSO. For intraperitoneal injections, 5, 10 and 30 mg/ml HC-030031 was suspended in 0.5% methylcellulose (Methocel, Fluka, Switzerland).

The TRPA1 antagonist referred to herein as “HC-030031” has the following formula:

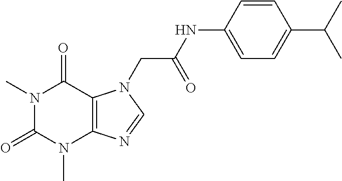

Ca²⁺-Imaging and Electrophysiology

Cultured neurons and HEK293t cells were loaded in modified Ringer's with 10 μM Fura-2-AM (Calbiochem, San Diego, Calif.) and 0.02% Pluronic F127 (BASF, Mount Olive, N.J., USA) for 1 hr, subsequently washed and imaged in glucose-free modified Ringer's. Fura-2 emission ratios were obtained with alternating 0.100 ms exposures at 340 and 380 nm from a Polychrome V monochromator (Till Photonics, Grafelfing, Germany) on a microscope (IX51, Olympus, Center Valley, Pa., USA), captured with a PCO camera (Sensicam QE, Cooke, Auburn Hills, Mich., USA) and analyzed with Imaging Workbench 6 software (Indec, Santa Clara, Calif., USA). Intracellular calcium ([Ca²⁺]ᵢ) concentrations were derived from the F₃₄₀/F₃₈₀ ratio adjusted by the K_{D} of Fura-2 (238 nM) and the F₃₈₀ and ratiometric data at minimum and maximum [Ca²⁺]ᵢ (48-50). The latter was determined by incubation in 10 □M ionomycin Ringer's solution with [Ca²⁺]ᵢ 10 mM EGTA or 25 mM Ca²⁺ (NaCl of 90 mM to compensate for a final osmolarity of 350 mOsm). Ratiometric images were generated using ImageJ software (http://rsbweb.nih.gov/ij/).

Whole-cell configuration patch-clamp experiments were performed at ˜25° C. with Borosilicate glass pipettes (WPI, World Precision Instruments, Inc., Sarasota, Fla., USA) for neurons and a planar patch clamp system for HEK-293t (NPC-1, Nanion, Munich, Germany). High-resolution currents were filtered at 2.3 kHz and digitized at 100 us intervals using an EPC-10 amplifier (HEKA, Lambrecht, Germany) and the PULSEMASTER acquisition software (HEKA). Voltage ramps of −100 to +100 mV or −80 to +80 mV were applied over 100 ms at every 0.5 Hz from a holding potential of 0 mV as previously described (51). Liquid junction potential of 6.7 mV (JPCalc software, Axon Instruments, MA, USA) and capacitance were compensated for by the amplifier system.

Single-channel patch-clamp experiments were performed in the cell-attached or inside-out configurations on CHO cells at ˜25° C. with wax-coated Borosilicate glass pipettes (WPI). High-resolution currents were filtered at 3 kHz and digitized at 20 μs intervals using an EPC-10 amplifier and the PULSEMASTER acquisition software (HEKA, Lambrecht, Germany).

Analysis of Nocifensive Responses

The nocifensive responses were examined in Trpa1^{-/-} and Trpa1^{+/+} mice to intraocular instillation of 10 μl of 100 mM or 200 mM HDI, 100 mM CN or 100 mM CS into the right eye or vehicle control (70% DMSO saline) into the left eye were video recorded (DCR-SR80, Sony, USA) in a clear Plexi-glass cylinder (5″ ID) for 2 or 3 min. At the conclusion of every test, the treated eye was irrigated with PBS saline. Mice responded to HDI or tear gas agent application by lowering and subsequent pushing or rubbing of the facial area on the floor of the behavioral recording chamber, which were individually counted. Nearly identical experiments were conducted on C-57 mice after 200 μl intraperitoneal (i.p.) injection of 0.5% vehicle control (100 mM HDI, 100 mM CN or 100 mM CS intraocular instillation into the right eye) and ˜1 hour after a 200 μl intraperitoneal injection of the TRPA1-antagonist HC-030031 (1 or 6 mg) (100 mM HDI, 100 mM CN or 100 mM CS into the right eye).

Nocifensive responses in the paw were also examined by 25 μl intraplantar injections using a 30 G needle. For experiments of Trpa1^{-/-} and Trpa1^{+/+} mice vehicle was injected into the left paw and then ˜1 hour later either CN (2 mM in 5% DMSO saline) or bromoacetone (4 mM in PBS) were injected in the right paw. For experiments using HC-030031 on C-57 mice, HDI (6 mM in 5% DMSO saline) or CN (4 mM in 5% DMSO saline) was injected into the right paw following 200 μl i.p. injection of 0.5% vehicle control and ˜1 hr after mice received a 2 mg HC-030031 i.p. injection (200 μl), 6 mM HDI or 4 mM CN was injected into the left paw. Video recorded responses (licking, lifting and flicking of the injected paw) in a Plexiglas cylinder for 3-5 min were visualized and quantified by slowing the video frame speed using Microsoft Windows Media Player software. The more hydrophobic agents were not used, because they were insoluble in 5% DMSO.

Statistics

Statistical analysis and graphical display of both electrophysiological and Ca²⁺-imaging data were made using IGOR PRO (Wavemetrics, Lake Oswego, Oreg., USA) or ORIGIN (OriginLab, Northampton, Mass., USA). Statistical errors are standard error of the mean (SEM) unless indicated otherwise.

An independent two-sample Student's t-test was conducted between mice lacking a functional Trpa1 gene (Trpa1^{-/-}) and wild-type littermates (Trpa1^{+/+}) on the total quantity of paw licking, lifting and flicking (paw pain response, n=8/group for CN and n=6/group for bromoacetone) and the “facial pain” of stroking the orbitofacial area in response to isocyanate or tear gases (CS and HDI n=6 Trpa1^{+/+}, n=7. Trpa1^{-/-}, n=6/group for CN). Differences were seen in the paw response to CN, p=0.023 and BrAc, p=0.045. Differences were seen in the “facial pain” response to CN, p=0.001, CS, p=0.001 and HDI, p=0.008.

Dependent (repeated measure) Student's t-test was conducted on the mouse “facial pain” and paw pain responses to isocyanate or tear gases after vehicle control injection compared to the responses ˜1 hour after the mice were injection with 6 mg HC-030031 (n=6/group for CS and HDI, n=9 for CN) or 1 mg HC-030031 (n=6/group) or 2 mg HC-030031 (n=6/group). Differences were seen in “facial pain” by 1 mg HC-030031 treatment to applications of CN p=0.004, CS p=0.04 and HDI, p=0.01 and by 6 mg HC-030031 treatment to applications of CN p=0.0, CS p=0.005 and HDI, p=0.029. Differences in paw pain were observed after 2 mg HC-030031 treatment to intraplantar injections of CN p=0.005 and HDI p=0.015.

Results

TRPA1 is Activated by Industrial Isocyanates and all Major Tear Gas Agents in Vitro.

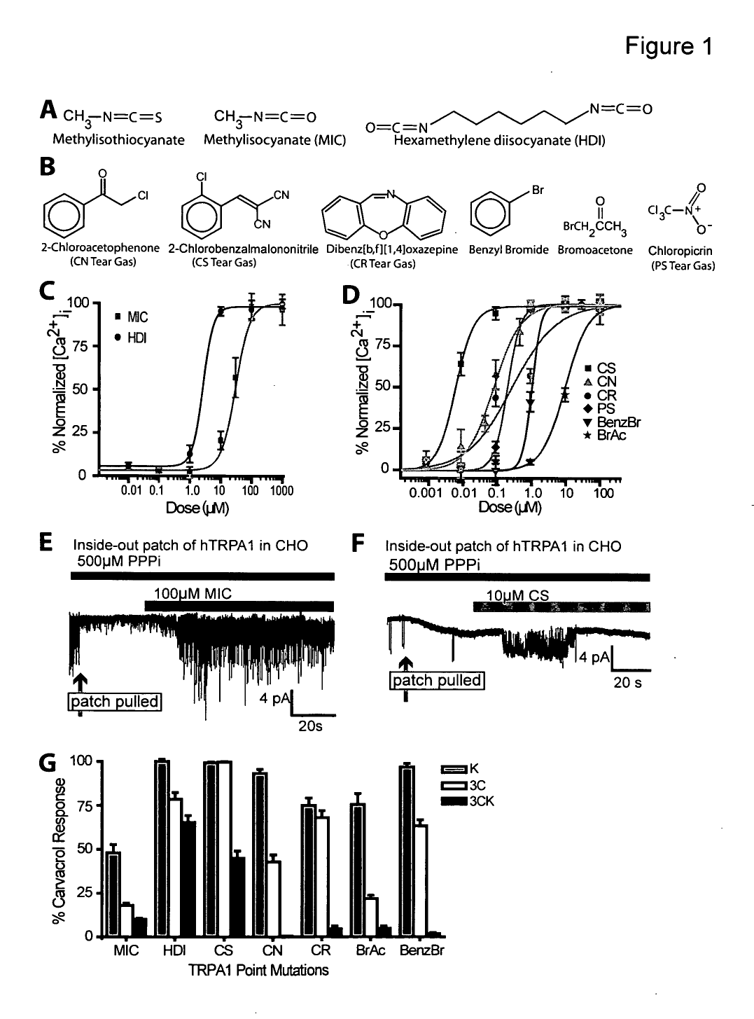

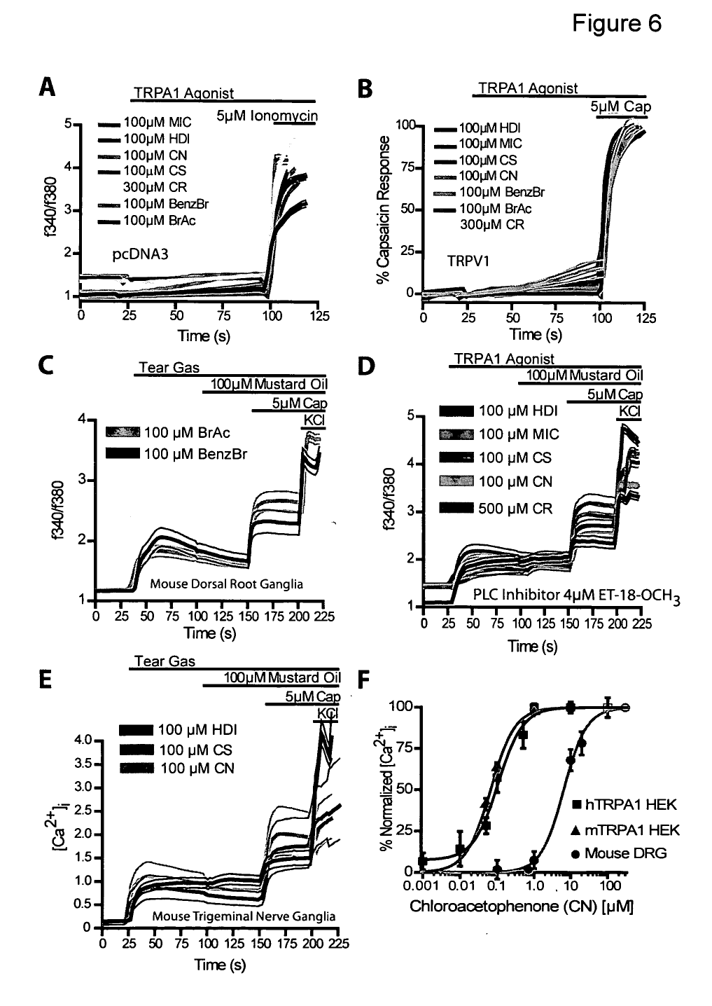

Fluorescent [Ca²⁺] imaging was used to examine the effects of two major industrial isocyanates (FIG. 1A) and six different tear gas agents (FIG. 1B) on two members of the Transient Receptor Potential (TRP) ion channel family, TRPV1, the capsaicin receptor, and TRPA1, the mustard oil receptor, expressed in human embryonic kidney cells (HEK293t). TRPA1 was strongly activated by MIC, HDI, and all the tear gas agents tested (CN, CS, CR, PS, bromoacetone and benzyl bromide). Dose response analysis revealed that the isocyanates MIC (EC₅₀=25±3 n=28±5 cells/dose) and HDI (EC₅₀=2.6±0.7 μM, n=29±4 cells/dose) activated TRPA1 with a potency comparable to the chemically similar mustard oil (allyl isothiocyanate)(FIG. 1C). In our hands, CS was the most potent activator of human TRPA1 channels with half maximal activation occurring at EC₅₀(CS)=7±1 nM (n=52±5 cells/dose) and three orders of magnitude more potent than mustard oil. CN, CR and PS were also highly potent with half maximal activation of human TRPA1 at EC₅₀(CN)=91±12 nM (n=49±1 cells/dose), EC₅₀(CR)=308±150 nM (n=70±9 cells/dose) and EC₅₀(PS)=308±150 nM (n=32±4 cells/dose) (FIG. 1D). The tear gas agents benzyl bromide (EC₅₀=12.0±0.6 n=66±17 cells/dose) and bromoacetone (EC₅₀=1.1±1.1 μM, n=78±14 cells/dose) also activated hTRPA1 At saturating doses of the noxious chemicals activation of hTRPA1, neither rat TRPV1 nor empty vector (pcDNA3) transfected HEK-293t cells responded (FIGS. 6A and 6B). Only HDI and benzyl bromide induced minor TRPV1 activity after significant delays following irritant application (FIG. 6B).

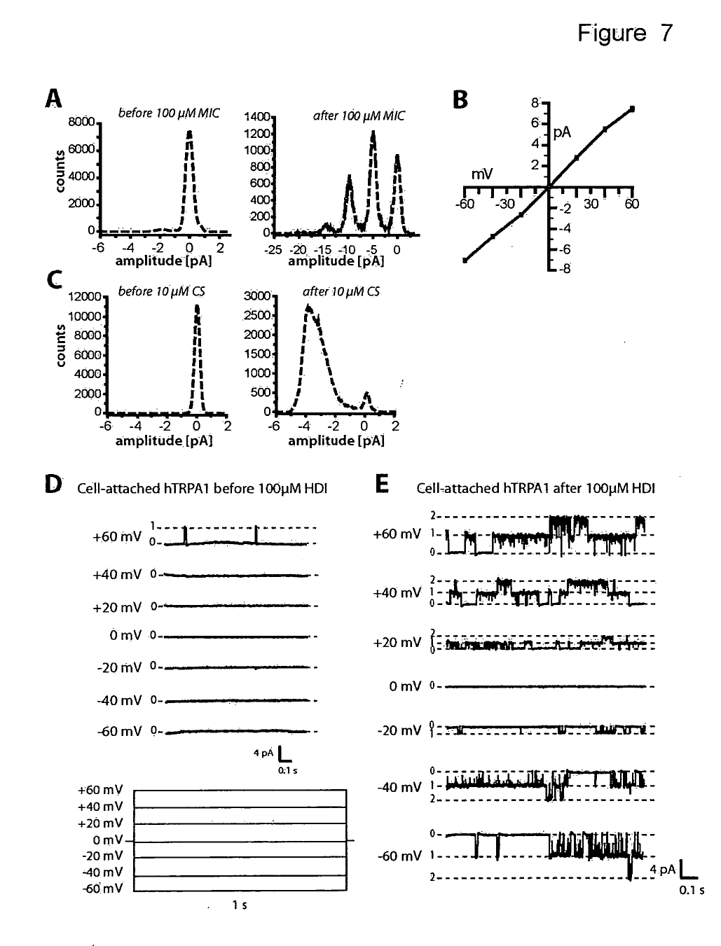

Recent studies support the idea that reactive irritants activate TRPA1 through covalent modification of cysteine and lysine residues within the cytosolic N-terminus of the channel protein (46, 47). While isocyanates and some tear gas agents can undergo electrophilic chemical reactions, CN, CS and CR also share structural similarities with non-reactive TRPA1 agonists, including terpenes such as carvacrol or thymol (44, 52, 53). Chemical agents may also activate TRPA1 indirectly, through stimulation of phospholipase C-coupled receptor pathways and subsequent release of Ca²⁺ from intracellular stores, or through other Ca²⁺-mobilizing pathways (29, 30, 54-56). To examine the requirement for Ca²⁺ or other cytosolic factors we performed inside-out patch-clamp recordings of hTRPA1 channels expressed in CHO-K1 cells, in the absence of Ca²⁺ on both sides of the membrane. In this configuration, PLC- and any other second messenger-dependent pathways are disrupted. Sodium triphosphate (0.5 μM), an essential intracellular co-factor for TRPA1 activation, was included in the bath solution (57). Application of 100 μM MIC or 10 μM CS specifically induced a large increase in single channel openings (124±3 pS for MIC and 120±3 pS for CS at −40 mV; 3 patches/agent), similar to TRPA1 single conductances recorded by others in the absence of Ca²⁺ (FIG. 1E, F, FIG. 7A, B) (58). These results suggest that isocyanates and tear gas agents activate TRPA1 in a membrane-delimited fashion that does not require increases in cytosolic Ca²⁺ or activation of second messenger pathways. hTRPA1 single channels were also activated in the cell-attached configuration, indicating that the chemical activator needs to traverse the plasma membrane to activate the ion channels positioned under the patch electrode (FIG. 7D, E). The open channel current-voltage relationship of HDI-activated channels in the cell attached configuration was linear in the absence of Ca²⁺ (single channel conductance: 127±4 pS at −40 mV) but outwardly rectifying in the presence 2 mM Ca²⁺ (51±2 pS at −40 mV) (FIG. 7D, E).

Next, it was examined whether isocyanates and tear gas agents would require putative covalent acceptor sites in hTRPA1 for channel activation (46, 47). Three different mutant channels were examined in which critical reactive sites (C619, C639, C663 and K708) were replaced by inert residues. In the first mutant (K) K708 was replaced. A second mutant (3C) had mutations in all three cysteine residues, and a third (3CK) mutations in all four sites. In previous studies these mutations dramatically reduced the potencies and efficacies of electrophiles and oxidants to activate TRPA1 (33, 36, 46, 47). As a positive control, the TRPA1 agonist carvacrol, a pungent non-reactive terpene, was used which does not activate TRPA1 by covalent binding. While the lysine mutant was activated by all agents (MIC n=84, HDI n=53, CS n=157, CN n=108, CR n=154, bromoacetone n=44 and benzyl bromide n=64 cells), mutant 3C showed significantly reduced responses to MIC (n=34), CN (n=56) and CR (n=90) and bromoacetone (n=25), but did not greatly affect the efficacy of HDI (n=47), CS (n=96) or benzyl bromide (n=40). CS, the most potent tear gas agent also showed significant activity on the 3CK mutant (n=129), as did the isocyanate HDI (n=123), indicating that these agents may require additional reactive sites for their activity, or activate TRPA1 through a different mechanism. In contrast, the activity of the other tested chemicals were dramatically reduced or eliminated (MIC (n=30), CN (n=18), CR (n=17), bromoacetone (n=38) and benzyl bromide (n=41)) (FIG. 1G).

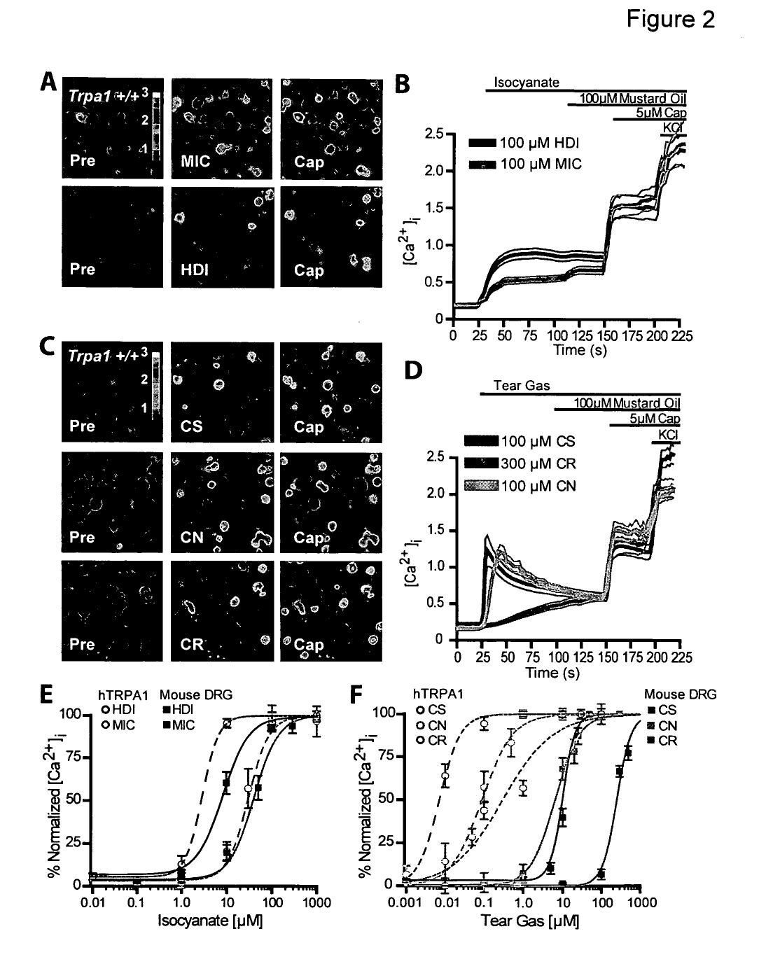

Cellular responses of native sensory neurons to industrial isocyanates have not been reported. Fluorescent Ca²⁺ imaging was used to investigate the effects of MIC and HDI on dissociated murine trigeminal (TG) and dorsal root ganglia (DRG) neurons. Fibers derived from the trigeminal ganglion innervate the eyes, facial skin and upper airways, which were the initial contact sites of exposure in patients during the Bhopal incident. DRG neurons innervate parts of the lower airways affected following inhalation of the toxicant. MIC (100 μM) and HDI (100 μM) were observed to induce a rapid increase in [Ca²⁺]ᵢ in a subset of capsaicin-sensitive TG and DRG neurons, overlapping with the mustard oil-sensitive neuronal population (FIG. 2A, B, FIG. 6E).

Responsiveness of native sensory neurons to the two most widely used tear gas agents, CS and CN, has not been described. CR was recently reported to activate Ca²⁺-influx into cultured DRG neurons (44). However, while implying TRPA1 as a neuronal target for CR, this study did not use any specific pharmacological, genetic or in vivo approaches to substantiate this point. CS, CN and bromoacetone and benzyl bromide (100 μM each) have now been found to rapidly induce Ca²⁺-influx into a subset of DRG neurons (FIG. 2C, D, F, FIG. 6C). Exposure to CS, CN, bromoacetone and benzyl bromide eliminated the neuronal sensitivity to subsequent application of mustard oil. CR (300 μM) only slowly induced neuronal activity and did not completely prohibit further neuronal activation by mustard oil (FIG. 2D). CS and CN also induced Ca²⁺ influx into TG neurons (FIG. 6F).

Similar to previously characterized TRPA1 agonists such as mustard oil or acrolein, the isocyanates have very similar potencies in mustard oil-sensitive DRG neurons (Ec₅₀ MIC-36±7 μM, n=30±6 neurons/dose) and (EC₅₀HDI=8.4±1.4 μM, n=39±12 neurons/dose) and in hTRPA1-transfected cells (EC₅₀ MIC=25±3 μM) and HDI (EC₅₀ HDI=2.6±0.7 μM) (FIG. 2E). Most surprisingly, it was found that the tear gas agents CS and CR were approximately 1.000-fold less potent, and CN to be 100-fold less potent, for activating Ca²⁺ influx into native neurons (EC₅₀CS=12.1±0.3 μM, n=41±9 neurons/dose, EC₅₀ CN=6±1 μM, n=23±5 neurons/dose, EC₅₀ CR=246±27 μM, n=37±16 neurons/dose) when compared to heterologous cells expressing hTRPA1 (EC₅₀ CS=7±1 nM, EC₅₀ CN=91±12 nM, EC₅₀ CR=308±150 nM) (FIG. 2F) or mouse TRPA1 (EC₅₀ CN=66±14 nM) (FIG. 6F).

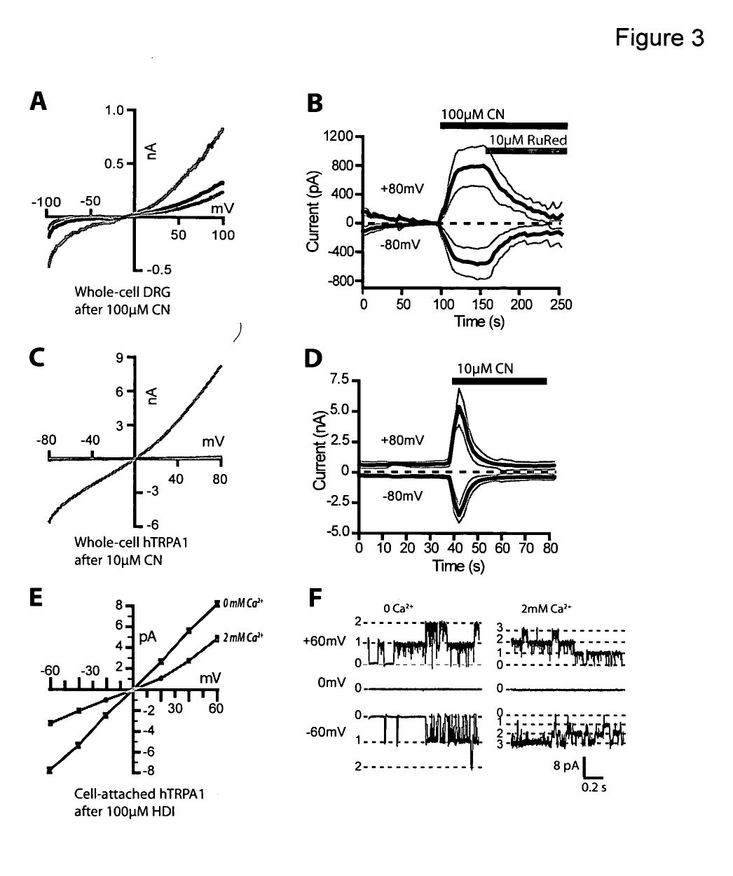

The large divergence of tear gas agent potencies between heterologous cells expressing TRPA1 and primary neurons suggests that either native TRPA1 channels have different pharmacological properties, or that alternative targets may be involved in neuronal responses to these agents. To further examine the neuronal response to tear gas agents, patch-clamp electrophysiological recordings of primary neurons in the whole-cell configuration was performed. CN (100 μM) induced sizable, slightly outwardly rectifying membrane currents in 4 out of 16 recorded neurons, which were efficiently blocked by ruthenium red, a pore blocker of TRPA1 and other TRP ion channels (FIG. 3A, B). The percentage of responsive neurons, the size and the current-voltage (I-V) relationship of the CN-induced currents were similar to neuronal TRPA1 currents we recorded in previous studies using the TRPA1 agonists sodium hypochlorite and isovelleral (33, 36). Furthermore, the CN-induced neuronal currents were remarkably similar in relationship to voltage as CN-induced currents in hTRPA1-expressing HEK293t cells (FIG. 3C). Compared to neuronal currents, TRPA1 currents in the heterologous HEK-293t were larger and desensitized rapidly, as characterized by us and others with a variety of agonists (n=4) (FIG. 3D) (36).

Genetic Deletion of TRPA1 or Pharmacological Blockade with TRPA1 Antagonists Renders Sensory Neurons Insensitive to Isocyanates and Tear Gas Agents

The results gathered from cultured primary neurons and heterologous cells suggest that industrial isocyanates and tear gas agents excite sensory neurons through activation of TRPA1. However, concentrations of tear gas agents required to induce Ca2+-influx into cultured sensory neurons were >100 fold higher than required for activation of cloned mouse and human TRPA1 channels expressed in heterologous cells. It remained a possibility that isocyanates and tear gas agents activated alternative targets in sensory neurons, through direct interactions with other Ca²⁺-permeable ion channels with relatively similar electrophysiological profiles, or indirectly, through activation of signal transduction cascades involving phospholipase C(PLC). PLC pathways have been shown to activate or sensitize TRPA1 and many other Ca²⁺-permeable TRP ion channels (26, 59). To investigate the potential involvement of PLC pathways in the neuronal response to isocyanates and tear gases, we performed Ca²⁺-imaging experiments in the presence ET-18-OCH₃, a PLC-inhibitor used in a previous study to inhibit activation of TRPA1 through PLC-coupled protease-activated receptors (PAR) in sensory neurons (56). ET-18-OCH₃ (4 □M) did not diminish neuronal Ca²⁺-influx activated by any of the noxious agents applied (Supplementary FIG. 1D).

To examine the requirement for TRPA1 in sensory neuronal responses to isocyanates and tear gas agents, the responses of sensory neurons dissociated from TRPA1-deficient mice were studied. When superfused with MIC (n=217 neurons from 2 mice), HDI (n=204 neurons from 2 mice), CS (n=229 neurons from 2 mice), CN (n=270 neurons from 5 mice) or CR (n=108 neurons), TRPA1-deficient neurons failed to respond with an increase in [Ca²⁺]ᵢ. These neurons responded normally to capsaicin, used as a control stimulus (FIG. 3A-D).

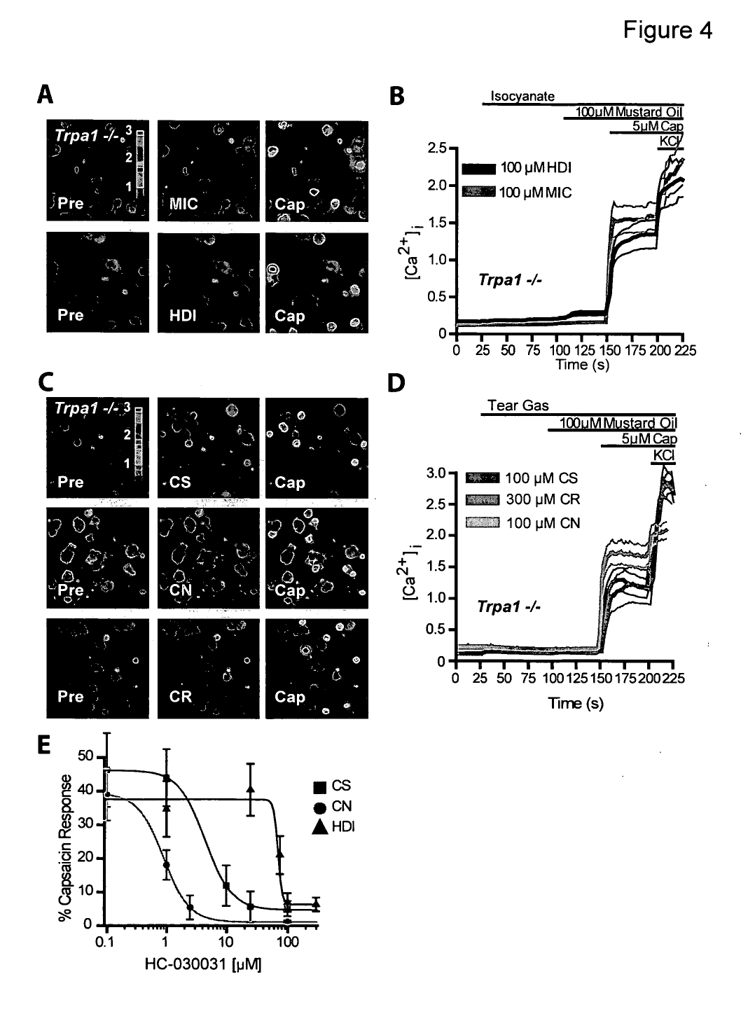

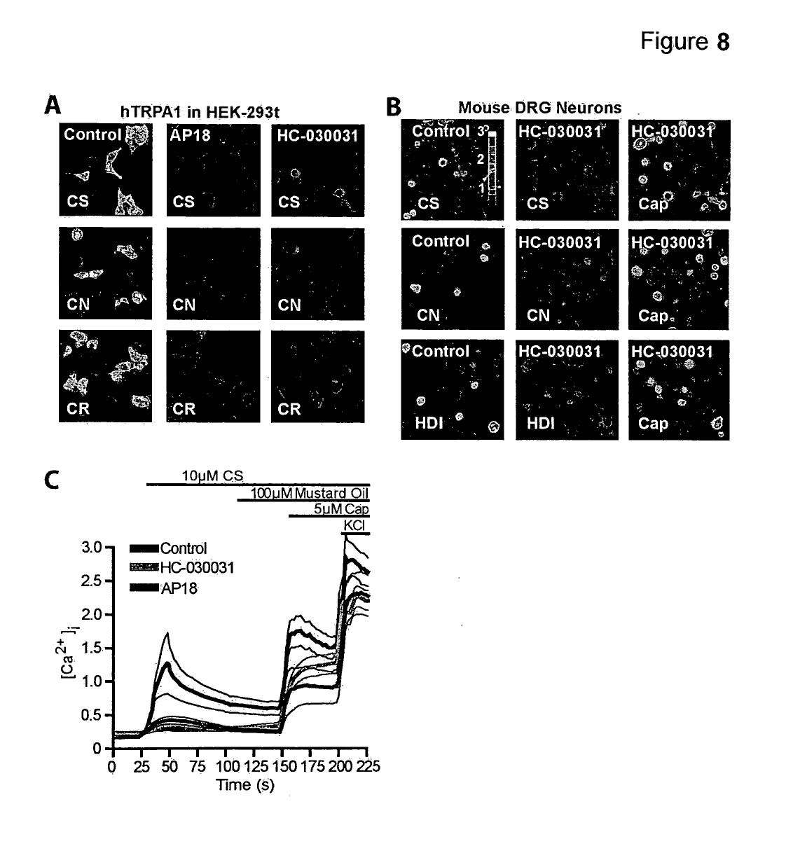

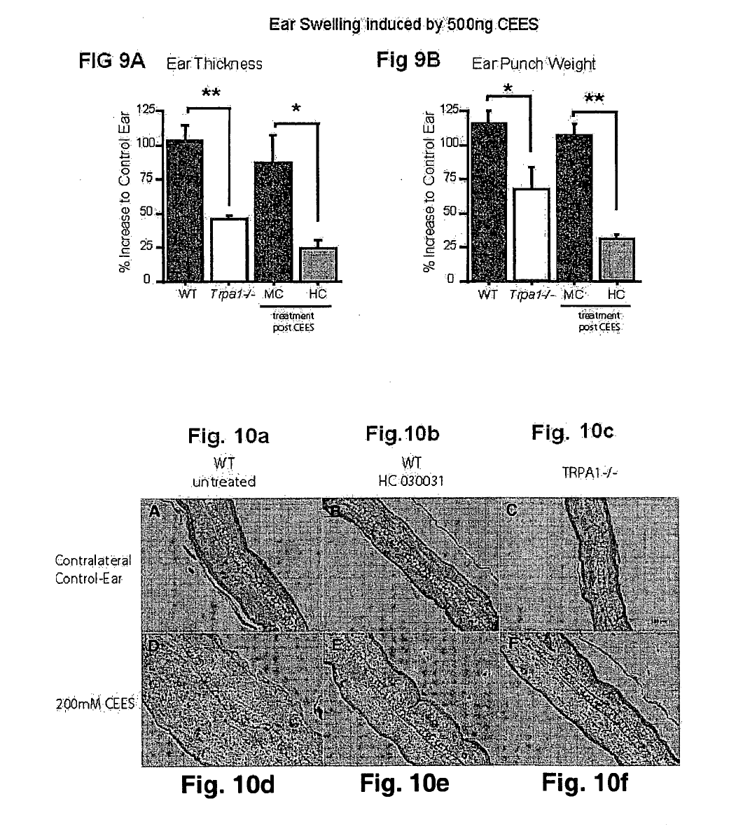

Recently, the structures and efficacies of two newly developed TRPA1 antagonists were reported (35, 60). These antagonists, HC-030031 and AP-18, blocked the activation of TRPA1 by mustard oil and other reactive chemical stimuli in vitro. The effects of these antagonists on CS-, CN- and CR-induced activation of hTRPA1 expressed in HEK293t cells were studied. Both HC-030031 and AP-18, used at a concentration of 25 μM, efficiently blocked the activation of hTRPA1 by all three tear gas agents (FIG. 8A). The antagonist HC-030031 effectively blocked native TRPA1 responses to 10 μM HDI (IC₅₀=74±3 μM, n=31±4), CN (IC₅₀=884±23 nM, n=25±5), and CS (IC₅₀=4.5±0.4 μM, n=26±6), in cultured sensory neurons dissociated from wild-type mice (FIG. 4D, FIGS. 8B and C). These neurons responded normally to a saturating dose of capsaicin, used as a control stimulus.

Taken together, the results show that TRPA1 is the sole target of industrial isocyanates and tear gas agents in sensory neurons, allowing influx of Ca²⁺ and neuronal excitation, and furthermore, that TRPA1 antagonist completely block neuronal activity in response to isocyanates or tear gas agents. This supports that TRPA1 antagonists may prevent and alleviate the noxious effects of isocyanates and tear gas agents in vivo.

TRPA1 Antagonists Effectively Block the Noxious Effects of Isocyanates and Tear Gas Agents In Vivo

Human exposure to airborne industrial isocyanates and tear gases results in immediate extreme ocular and facial pain, as well as airway irritation, mucus secretion and obstruction. The data suggests that these effects are triggered by activation of TRPA1 channels in trigeminal sensory neurons. However, it is unclear whether isocyanates and tear gas agents interact specifically with TRPA1 in vivo, or if these highly reactive chemicals activate sensory neurons indirectly through factors released during tissue damage, and so the effects of pharmacological inhibition and genetic ablation of TRPA1 on the behavioral responses to isocyanates and tear gas agents in mice were examined.

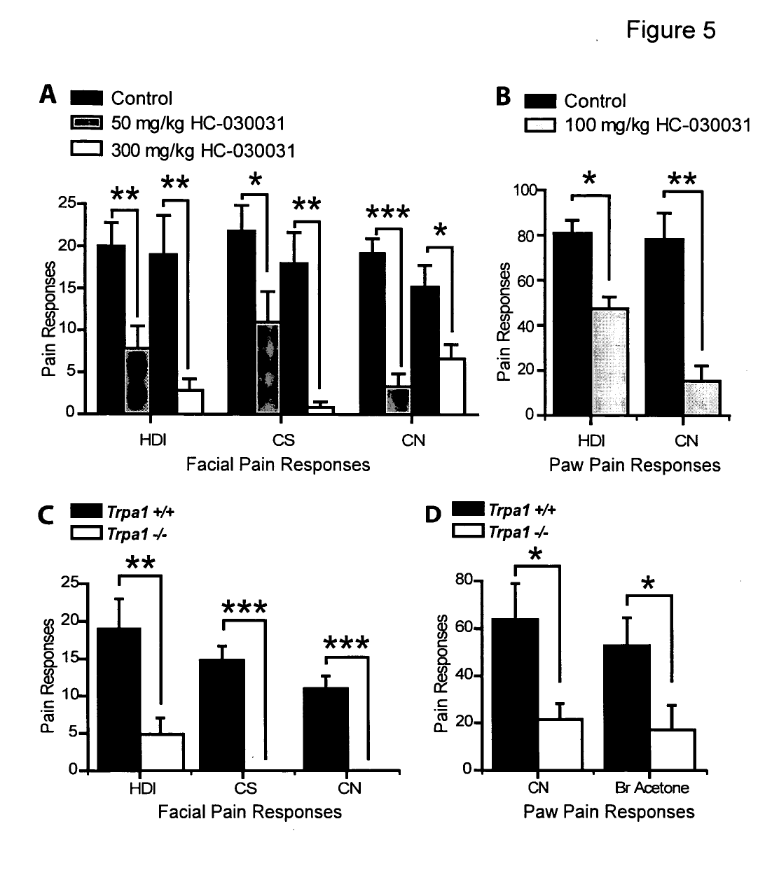

HDI, CN, and CS (100 mM each) caused immediate nocifensive responses upon application to the mouse eye (MIC was too volatile and dangerous to test). The mice initially wiped their eyes and facial area, and then continued with characteristic nocifensive behavior by vigorously stroking their heads and facial area against the bottom of the observation chamber (33). This behavior was completely absent when just vehicle was applied. We then injected the mice with the TRPA1 antagonist HC-030031 (300 mg/kg BW or 50 mg/kg BW, i.p.) and applied the same dose of noxious chemical to the opposite eye one hour later (300 mg/kg HC-030031 (n=6/group for CS and HDI, n=9 for CN) and 50 mg/kg HC-030031 (n=6/group). Remarkably, HC-030031 dramatically reduced the frequency of nocifensive responses to all three agents (FIG. 5A). A more conventional method of examining TRPA1-associated nocifensive responses, was then used, comparing nocifensive responses following intraplantar injections of HDI (6 mM) or the tear gas agent CN (4 mM) into the mouse hindpaw before and after treatment with 100 mg/kg BW HC-030031.

Following the initial intraplantar injections, mice responded with immediate nocifensive behavior, including flinching, lifting and licking of the paw (FIG. 5B). This behavior was greatly reduced in the same mice approximately one hour after treatment with HC-030031 (FIG. 5B).

Since HC-030031 may inhibit the effects of isocyanates and tear gases in a non-specific manner, isocyanate- and tear gas agent-induced behavior between TRPA1-deficient mice following eye application was also compared. Strikingly, nocifensive responses to tear gas agents (CN and CS) were completely absent in Trpa1^{-/-} mice in this test (FIG. 5C). These results suggest that Trpa1−/− mice fail to detect tear gas agents as noxious stimuli. Responses to the isocyanate HDI were significantly abated (FIG. 5C). In addition to facial exposures, responses of Trpa1^{-/-} and Trpa1^{+/+} mice following injections of the relatively soluble tear gas agents CN (n=8/group) and bromoacetone (n=6/group) into the hindpaw were compared. Following injections, wild-type mice responded with immediate nocifensive behavior, which was greatly reduced in Trpa1^{-/-} mice (FIG. 5D).

In summary, these behavioral tests support an essential role for TRPA1 in the sensory detection of industrial isocyanates and tear gas agents (CN, CS and bromoacetone) in vivo. Furthermore, exposure-related pain and irritation by these agonists can be prevented by administering TRPA1 antagonists prior to exposure.

### DISCUSSION

The above testing has demonstrated that industrial isocyanates target the same sensory neuronal receptor as tear gas agents, TRPA1, to rapidly activate pain and sensory irritation. Thus, TRPA1 channels expressed in primary sensory neurons and heterologous cells are robustly activated by both classes of agents. However, isocyanate and tear gas-induced nocifensive behavior is greatly reduced in TRPA1-deficient mice, and the treatment of mice with TRPA1 antagonists leads to a dramatic reduction in sensitivity to isocyanates and tear gas agents.

Activation of TRPA1 by industrial isocyanates may have contributed to the acute and chronic health effects experienced by victims of the Bhopal incident, agricultural and industrial laborers (1, 6). It was found that the industrial isocyanates strongly activate human TRPA1 channels and, in mice, have effects very similar to tear gases, activating trigeminal nerve endings in the eyes and facial area to elicit nocifensive responses. Trigeminal nerve fibers innervating the facial skin, mucous membranes and eyes are the first line of defense against chemical exposures threatening tissue integrity and function (22). By acting similar to tear gas agents, isocyanates induce ocular pain, lacrimation and blepharospasm through trigeminal-autonomic and trigeminal-motor reflexes in exposed individuals. In addition to ocular and facial cutaneous nerve endings, isocyanates may also target TRPA1 channels in nerve endings lining the airways. In humans, activation of airway nerve endings by chemical irritants triggers cough, sneezing, airway mucus secretion, edema and obstruction through activation of sensory nerves. In mice, these effects result in respiratory depression, significantly lowering respiratory rates (61).

It was found that TRPA1 is essential for the activation of murine sensory neurons by the irritant chlorine, and for chlorine-induced respiratory depression (36). Similar to chlorine, isocyanates and other TRPA1 agonists such as acrolein induce respiratory depression in rodents and other mammalian species, suggesting a crucial role of TRPA1 in this physiological response to chemical sensory irritation (14, 62, 63).

The above testing has provided a clear mechanistic basis for the biological actions of tear gases in vivo, supporting a central role of TRPA1 in the neuronal sensation of all major tear gas agents and subsequent activation of involuntary nocifensive reflex responses, including lacrimation, mucus secretion and muscle contraction, with CS and CN identified as the most highly potent activators of heterologously expressed human TRPA1 channels. It was found that CR is less potent than CS and CN, a finding which is in contrast to a recent study that reported a higher potency of CR on hTRPA1 in vitro (44). The reason for this discrepancy may lie in the differing purity of the agents used, or in differences in experimental conditions.

Large differences in potencies of tear gas agents in heterologous cells and native sensory neurons were observed. While divergence of potencies have been observed for TRPA1 agonists before, it was found that some tear gas agents have >100-fold higher potencies in human or mouse TRPA1-expressing HEK293t cells than in mouse sensory neurons (36). In contrast, isocyanates show largely equal potencies in heterologous cells and native neurons. The results indicate that in vitro studies alone are insufficient to evaluate specific TRPA1 agonist activity for a given chemical.

It was also found that previously identified covalent acceptor sites in TRPA1 are essential for activation by some agonists (CN, CR), but not by others (MIC, HDI, CS). These results suggest that, in addition to electrophilic reactivity, other factors affect the ability of give chemical agents to activate TRPA1. Some chemical agonists may bind to additional, as yet unidentified, covalent acceptor sites. Other agents may have different membrane permeabilities in heterologous cells or neurons, or their actions may be affected by intracellular reducing agents. Finally, responses by native TRPA1 channels may be affected by additional protein subunits, post-translational modifications, or differences in regulation of the local Ca²⁺ microenvironment (64).

The essential role of TRPA1 as the sole mediator of tear gas-related irritation in vivo is supported by the observation that TRPA1-deficient mice are largely impervious to the noxious effects of tear gases. In contrast to isocyanates, exposure to tear gas agents causes less tissue damage and long-term health effects. CS and CN are much less volatile than MIC, and are usually dispersed as aerosols together with organic solvents or burned to reach irritating airborne concentrations (12). Nevertheless, adverse health effects, and even deaths, have been reported following tear gas exposures, especially when exposures occurred in closed environments. Responses include acute bronchospasm, pulmonary edema, asthma-like symptoms and severe contact dermatitis (65-70).

Individuals affected by pre-existing allergic conditions seem to be especially prone to hypersensitivity reactions following tear gas exposures. In addition to the two major tear gas agents, TRPA1 is also activated by CR, benzyl bromide, bromoacetone and chloropicrin (PS). Presently, chloropicrin is widely used as a soil fumigant in agriculture, causing frequent occupational and environmental exposures (71, 72). TRPA1 activation is likely to contribute to the health effects caused by chloropicrin, including eye and respiratory tract irritation.

Irritant-induced sensory reflexes and pain are thought to be essential for the protection of eyes, skin and airways from further chemical exposures. However, in the cases of isocyanates and tear gases, sensory responses usually occur rapidly and with very high intensity, leading to partial or complete incapacitation. During the Bhopal incident, the TRPA1-mediated acute noxious effects of methyl isocyanate may thus have prevented many victims from escaping further exposure, leading to aggravated tissue damage due to the non-specific corrosive effects of the toxicant. Individuals suffering from airway infections or chronic inflammatory airway conditions, both highly prevalent in developing countries, may have responded more violently to MIC exposure. Activation of inflammatory signaling pathways in asthma, rhinitis or airway infections could explain hypersensitivity responses to isocyanates and tear gases, since these pathways dramatically increase the sensitivity of TRPA1 to its agonists (9, 29, 30, 34, 56).

Individuals exposed to high levels of TRPA1 agonists, including chlorine and isocyanates, often present with reactive airways dysfunction syndrome (RADS) (73-75). RADS is characterized by highly increased sensitivity to chemical and physical stimuli, in addition to the initial sensitizing stimulus, resulting in asthma-like symptoms such as cough, wheezing, chest tightness and dyspnoea (73). For example, agricultural workers exposed to MIC during a spill of the pesticide, metam sodium, subsequently became highly sensitive to diesel exhaust (5). Diesel exhaust contains high levels of the TRPA1 agonist, acrolein, and induced lacrimation, strong nasal irritation and cough in the MIC-preexposed individuals (5). The multiple chemical sensitivity of TRPA1 readily explains the symptoms observed in RADS patients. Following initial sensory challenge and tissue injury by a high-level chemical exposure, sensory TRPA1 channels become sensitized through inflammatory signaling pathways, establishing prolonged hypersensitivity to multiple reactive chemicals (29, 30, 34, 56). The role of TRPA1 in chemical hypersensitivity may extend to other, less clearly defined, conditions, including sensory hyperreactivity (SHR) and multiple chemical sensitivity (MCS) (76, 77).

RADS and related conditions are only partially responsive to the therapeutic interventions developed for the treatment of asthma. The data supports a method using TRPA1 antagonists to effect blocking of the exaggerated chemosensory responses accompanying these conditions. Moreover, administering the TRPA1 antagonists prevents the acute sensory irritation elicited by exposures to isocyanates and tear gasses. Moreover, administering TRPA1 antagonists is believed to be useful for post-exposure treatment, reducing sensory irritation and, potentially, preventing adverse long-term health effects elicited by neurogenic inflammatory mechanisms.

In certain embodiments, the present invention provides a method for treating or reducing the likelihood of a condition involving activation of TRPA1 in response to toxicant exposure or for which reduced TRPA1 activity can reduce the severity of the effects from the exposure. There are a number of known compounds useful in the performance of the method of the invention, a number of which are disclosed in US patent application publication no. US2007 0219222, entitled “Methods and Compositions for Treating Pain”, published Sep. 20, 2007, which is incorporated by reference in its entirety herein. For example, the invention may comprise administering an effective amount of a compound of Formula I or a salt thereof, or a solvate, hydrate, oxidative metabolite or prodrug of the compound or its salt:

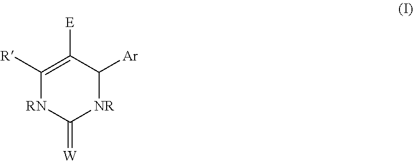

wherein W represents O or S, preferably S; R, independently for each occurrence, represents H or lower alkyl, preferably H; R′ represents substituted or unsubstituted alkyl or substituted or unsubstituted aryl; E represents carboxylic acid (CO₂H), ester or amide; and Ar represents a substituted or unsubstituted aryl ring, or comprise administering an effective amount of a compound of Formula II or a salt thereof, or a solvate, hydrate, oxidative metabolite or prodrug of the compound or its salt:

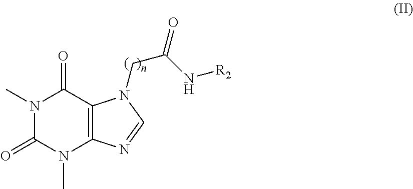

wherein n is an integer from 1 to 3; and R₂ represents a substituent, which is optionally substituted alkyl, optionally substituted aryl, optionally substituted heteroaryl, optionally substituted aralkyl, optionally substituted cycloalkyl, optionally substituted heterocyclyl, or optionally substituted heteroaralkyl.

Alternatively, the method may comprise administering an effective amount of a compound of Formula III or a salt thereof, or a solvate, hydrate, oxidative metabolite or prodrug of the compound or its salt:

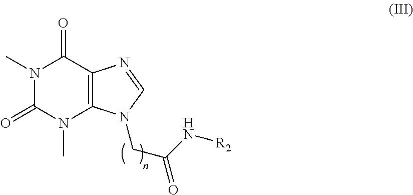

wherein n is an integer from 1 to 3; and R₂ represents a substituent, optionally substituted alkyl, optionally substituted aryl, optionally substituted heteroaryl, optionally substituted aralkyl, optionally substituted cycloalkyl, optionally substituted heterocyclyl, or optionally substituted heteroaralkyl.

Alternatively, the method may comprise administering an effective amount of a compound of Formula IV or a salt thereof, or a solvate, hydrate, oxidative metabolite or prodrug of the compound or its salt:

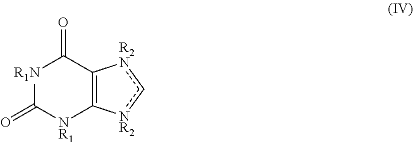

wherein R₁, independently for each occurrence, represents H or lower alkyl; one occurrence of R₂ is absent and one occurrence of R₂ is MₘR₃; R₃ represents substituted or unsubstituted aryl; M, independently for each occurrence, represents a substituted or unsubstituted methylene group (e.g., substituted with lower alkyl, oxo, hydroxyl, etc.), NR₁, O, S, S(O), or S(O₂), preferably selected such that no two heteroatoms are adjacent to each other; and m is an integer from 0-10, preferably where MₘR₃ represents:

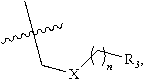

wherein n is an integer between 0 and 4; and X is or —C(═O)NR₄— wherein R₄ is H or lower alkyl, preferably —C(═O)NH—.

The TRPA1 inhibitor for use in methods or pharmaceutical preparations of the present invention may also comprise Ruthenium Red (ammoniated ruthenium oxychloride), having the following Formula V or a salt thereof, or a solvate, hydrate, oxidative metabolite or prodrug of the compound or its salt:

[(NH₃)₅RuORu(NH₃)₄ORu(NH₃)₅]⁶⁺6Cl⁻ V

or may comprise AP-18, 4-(4-Chlorophenyl)-3-methylbut-3-en-2-oxime, having the following Formula VI or a salt thereof, or a solvate, hydrate, oxidative metabolite or prodrug of the compound or its salt:

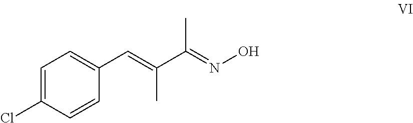

The present invention provides a method of administering an effective amount of any of the compounds shown above (e.g., a compound of Formula I, Formula II, Formula III, Formula IV, Formula V, Formula VI or a salt thereof, or a solvate, hydrate, oxidative metabolite or prodrug of the compound or its salt), as well as other TPVA1 antagonists, as a pharmaceutical preparation suitable for use in a human patient, or for veterinary use, and one or more pharmaceutically acceptable excipients in a method for preventing, reducing of inhibiting the noxious effects of exposure to toxicants. Pre-exposure administration acts as a prophylactic to prevent or inhibit the noxious effects, while post exposure administration can act as a treatment which would ameliorate the noxious effects. In particular, those suffering from chemical sensitivity may benefit from the method of the invention, to reduce the sensitivity to toxicants such as isocyanates and tear gas.

Kits containing the counteracting agents disclosed herein could be prepared and available for example when tear gas is or will be used, to either rapidly treat those exposed, such as those suffering from the exposure, particularly non-targeted civilians, children, law enforcement personnel, medical technicians, etc., as well as those for whom such exposure could be life threatening. Those who will enter an area where such toxicants has been or will be released can be administered the counteracting agents prior to exposure to prevent of lessen the effects of the exposure.

TRPA1 antagonists can be administered alone or in combination with other therapeutic agents. For instance, the TRPA1 antagonists may be administered with one or more of an anti-inflammatory agent, anti-scarring agent, anti-psoriatic agent, anti-proliferative agent, or anti-septic agent, among others.

The TRPA1 antagonists can be administered in any acceptable form, such as topically, orally, transdermally, rectally, vaginally, parentally, intranasally, intraocularly, intravenously, intramuscularly, intraarterially, intrathecally, intracapsularly, intraorbitally, intracardiacly, intradermally, intraperitoneally, transtracheally, subcutaneously, subcuticularly, intraarticularly, subcapsularly, subarachnoidly, intraspinally, intrasternally or by inhalation.

The terms “antagonist” and “inhibitor” are used interchangeably to refer to an agent that decreases or suppresses a biological activity, such as to repress an activity of an ion channel, such as TRPA1. TRPA1 inhibitors include inhibitors having any combination of the structural and/or functional properties disclosed herein. Also, “inhibit” as used herein refers to the partial or complete elimination of a potential effect, while inhibitors are compounds that have the ability to inhibit.

Pharmaceutical Compositions

The method of the invention contemplates the administration of a TRPA1 antagonist alone, but more preferably as a pharmaceutical composition, which can be formulated using know methods to adapt the TRPA1 antagonist for administration via known routes, such as topically, orally, transdermally, rectally, vaginally, parentally, intranasally, intraocularly, intravenously, intramuscularly, intraarterially, intrathecally, intracapsularly, intraorbitally, intracardiacly, intradermally, intraperitoneally, transtracheally, subcutaneously, subcuticularly, intraarticularly, subcapsularly, subarachnoidly, intraspinally, intrasternally or by inhalation. Thus, the compounds according to the invention may be formulated for administration in any convenient way for use in human or veterinary medicine, and formulated into pharmaceutically acceptable dosage forms such as described below or by other conventional methods known to those of skill in the art.

The method of the invention thus includes administering pharmaceutically acceptable compositions containing a therapeutically effective amount of a TRPA1 antagonist, which may be one or more of the compounds described above, formulated together with one or more pharmaceutically acceptable carriers and/or diluents. The pharmaceutical compositions may be formulated for administration in solid or liquid form, and adapted for oral administration, as aqueous or non-aqueous solutions or suspensions, tablets, boluses, powders, granules, pastes for application to the tongue or adapted for parenteral administration by subcutaneous, intramuscular or intravenous injection. Topical applications by way of a cream, ointment or spray may be of particular interest in countering the effects of toxicants, post exposure, as well as formulations that are administered via inhalation.

The phrase “therapeutically effective amount” as used herein means that amount of a compound, material, or composition which is effective for producing a desired therapeutic effect in response to exposure to a toxicant or chemical irritant by inhibiting TRPA1 function in at least a sub-population of cells in an animal and thereby blocking the biological consequences of that function in the treated cells, at a reasonable benefit/risk ratio applicable to any medical treatment.

“Pharmaceutically acceptable” as used herein means that the compound or composition is suitable for administration to a subject to achieve the treatments described herein, without unduly deleterious side effects in light of the severity of the disease and necessity of the treatment.

The phrase “pharmaceutically acceptable carrier, additive or excipient” as used herein means a pharmaceutically acceptable material, composition or vehicle, such as a liquid or solid filler, diluent, carrier, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient.

It should be understood that various additives, such as wetting agents, emulsifiers and lubricants, as well as coloring agents, release agents, coating agents, sweetening, flavoring and perfuming agents, preservatives and antioxidants can also be present in the compositions administered according to the present invention.

Formulations of the invention suitable for oral administration may be in the form of capsules, cachets, pills, tablets, lozenges, powders, granules, or as a solution or a suspension in an aqueous or non-aqueous liquid, or as an oil-in-water or water-in-oil liquid emulsion, or as an elixir or syrup, or as pastilles and/or as mouth washes and the like, each containing a predetermined amount of the TRPA1 antagonist as an active ingredient. Of course, the composition can be formulated so as to provide slow or controlled release of the active ingredient using known pharmacological procedures, such as, for example, hydroxypropylmethyl cellulose in varying proportions to provide the desired release profile.

Topical or transdermal administration may be by way of applying powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required. Ophthalmic formulations in the form of eye ointments, powders, solutions and the like, may also be used in the method of the invention.

The method of the invention contemplates the administration of the TRPA1 antagonists as pharmaceuticals, to humans and animals, administered per se or as a pharmaceutical composition containing, for example, 0.1 to 99.5% (more preferably, 0.5 to 90%) of the active ingredient in combination with a pharmaceutically acceptable carrier. Actual dosage levels of the active ingredient may be varied so as to obtain an amount of the active ingredient that is effective to achieve the desired therapeutic response from an individual patient, and given the choice of TRPA1 agonist, mode of administration, etc.

The actual dosage depends upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts, and it is within the ordinary skill of a physician to determine the effective dose.

In general, a suitable daily dose of a compound of the invention will be that amount of the compound that is the lowest dose effective to produce a therapeutic effect. Such an effective dose will generally depend upon the factors described above. Generally, doses will range from about 0.0001 to about 100 mg per kilogram of body weight per day.

The patient receiving this treatment is any animal in need, including primates, in particular humans, and other mammals such as equines, cattle, swine and sheep; and poultry and pets in general.

As discussed above, for example, the TVPA1 antagonists can be administered prophylactically to a mammal in advance of the exposure to the toxicant, such as tear gas. Prophylactic administration is effective to decrease the likelihood of the subsequent noxious effects of the exposure, such as occurrence of disease in the mammal, or decrease the severity of effects that subsequently occur, in particular, peripheral neuropathy, inducing either numbness or chronic neuropathic pain, reactive airways dysfunction syndrome (RADS), due to lung injury, blindness, due to eye inflammation, skin scarring, hyperpigmentation, folliculitis, pulmonary fibrosis, bronchiectasis, and pneumonia.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. Any inconsistency between the material incorporated by reference and the material set for in the specification as originally filed shall be resolved in favor of the specification as originally filed. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

### REFERENCES

1. Dhara, V. R., and Dhara, R. (2002) The Union Carbide disaster in Bhopal: a review of health effects. Arch Environ Health 57, 391-404
2. Koehler, G. A., and Van Ness, C. (1993) The emergency medical response to the Cantara hazardous materials incident. Prehosp Disaster Med 8, 359-365
3. Pruett, S. B., Myers, L. P., and Keil, D. E. (2001) Toxicology of metam sodium. J Toxicol Environ Health B Crit. Rev 4, 207-222
4. (1991) Dermatitis among workers cleaning the Sacramento River after a chemical spill—California, 1991. MMWR Morb Mortal Wkly Rep 40, 825-827, 833
5. Cone, J. E., Wugofski, L., Balmes, J. R., Das, R., Bowler, R., Alexeeff, G., and Shusterman, D. (1994) Persistent respiratory health effects after a metam sodium pesticide spill. Chest 106, 500-508
6. Varma, D. R., and Guest, I. (1993) The Bhopal accident and methyl isocyanate toxicity. J Toxicol Environ Health 40, 513-529
7. Woolrich, P. F. (1982) Toxicology, industrial hygiene and medical control of TDI, MDI and PMPPI. Am Ind Hyg Assoc J 43, 89-97
8. Gupta, B. N., Stefanski, S. A., Bucher, J. R., and Hall, L. B. (1987) Effect of methyl isocyanate (MIC) gas on the eyes of Fischer 344 rats. Environ Health Perspect 72, 105-108
9. Olajos, E. J., and Salem, H. (2001) Riot control agents: pharmacology, toxicology, biochemistry and chemistry. J Appl Toxicol 21, 355-391
10. Sartori, M., and Marrison, L. W. (1939) The war gases, chemistry and analysis, D. Van Nostrand, N.Y.,
11. Warden, C. R. (2005) Respiratory Agents: Irritant Gases, Riot Control Agents, Incapacitants, and Caustics. Critical Care Clinics 21, 719-737
12. Blain, P. G. (2003) Tear gases and irritant incapacitants. 1-chloroacetophenone, 2-chlorobenzylidene malononitrile and dibenz[b,f]-1,4-oxazepine. Toxicol Rev 22, 103-110
13. de Torres, J. P., Correa, V., Rosquete, J., and Febles, T. (2006) Riot control agents and their respiratory effects. Respiratory Medicine Extra 2, 13-15
14. Alarie, Y., Ferguson, J. S., Stock, M. F., Weyel, D. A., and Schaper, M. (1987) Sensory and pulmonary irritation of methyl isocyanate in mice and pulmonary irritation and possible cyanidelike effects of methyl isocyanate in guinea pigs. Environ Health Perspect 72, 159-167
15. Wisnewski, A. V., and Redlich, C. A. (2001) Recent developments in diisocyanate asthma. Curr Opin Allergy Clin Immunol 1, 169-175
16. Herrick, C. A., Xu, L., Wisnewski, A. V., Das, J., Redlich, C. A., and Bottomly, K. (2002) A novel mouse model of diisocyanate-induced asthma showing allergic-type inflammation in the lung after inhaled antigen challenge. J Allergy Clin Immunol 109, 873-878
17. Wisnewski, A. V., Liu, Q., Liu, J., and Redlich, C. A. (2008) Human innate immune responses to hexamethylene diisocyanate (HDI) and HDI-albumin conjugates. Clin Exp Allergy 38, 957-967
18. Gordon, T., Sheppard, D., McDonald, D. M., Distefano, S., and Scypinski, L. (1985) Airway hyperresponsiveness and inflammation induced by toluene diisocyanate in guinea pigs. Am Rev Respir Dis 132, 1106-1112
19. Ferguson, J. S., and Alarie, Y. (1991) Long term pulmonary impairment following a single exposure to methyl isocyanate. Toxicol Appl Pharmacol 107, 253-268
20. Thompson, J. E., Scypinski, L. A., Gordon, T., and Sheppard, D. (1987) Tachykinins mediate the acute increase in airway responsiveness caused by toluene diisocyanate in guinea pigs. Am Rev Respir Dis 136, 43-49
21. Mapp, C. E., Boniotti, A., Graf, P. D., Chitano, P., Fabbri, L. M., and Nadel, J. A. (1991) Bronchial smooth muscle responses evoked by toluene diisocyanate are inhibited by ruthenium red and by indomethacin. Eur J Pharmacol 200, 73-76
22. Mutch, J. R. (1944) The Lacrimation Reflex. Br J Ophthalmol 28, 317-336
23. Karagama, Y. G., Newton, J. R., and Newbegin, C. J. (2003) Short-term and long-term physical effects of exposure to CS spray. J R Soc Med 96, 172-174
24. Wang, H., and Woolf, C. J. (2005) Pain TRPs. Neuron 46, 9-12
25. Jordt, S. E., and Ehrlich, B. E. (2007) TRP channels in disease. Subcell Biochem 45, 253-271
26. Venkatachalam, K., and Montell, C. (2007) TRP channels. Annu Rev Biochem 76, 387-417
27. Caterina, M. J., Schumacher, M. A., Tominaga, M., Rosen, T. A., Levine, J. D., and Julius, D. (1997) The capsaicin receptor: a heat-activated ion channel in the pain pathway. Nature 389, 816-824
28. Caterina, M. J., Leffler, A., Malmberg, A. B., Martin, W. J., Trafton, J., Petersen-Zeitz, K. R., Koltzenburg, M., Basbaum, A. I., and Julius, D. (2000) Impaired nociception and pain sensation in mice lacking the capsaicin receptor. Science 288, 306-313
29. Jordt, S. E., Bautista, D. M., Chuang, H. H., McKemy, D. D., Zygmunt, P. M., Hogestatt, E. D., Meng, I. D., and Julius, D. (2004) Mustard oils and cannabinoids excite sensory nerve fibres through the TRP channel ANKTM1. Nature 427, 260-265
30. Bandell, M., Story, G. M., Hwang, S. W., Viswanath, V., Eid, S. R., Petrus, M. J., Earley, T. J., and Patapoutian, A. (2004) Noxious cold ion channel TRPA1 is activated by pungent compounds and bradykinin. Neuron 41, 849-857
31. Bautista, D. M., Movahed, P., Hinman, A., Axelsson, H. E., Sterner, O., Hogestatt, E. D., Julius, D., Jordt, S. E., and Zygmunt, P. M. (2005) Pungent products from garlic activate the sensory ion channel TRPA1. Proc Natl Acad Sci USA 102, 12248-12252
32. Macpherson, L. J., Geierstanger, B. H., Viswanath, V., Bandell, M., Eid, Hwang, S., and Patapoutian, A. (2005) The pungency of garlic: activation of TRPA1 and TRPV1 in response to allicin. Curr Biol 15, 929-934
33. Escalera, J., von Helm, C. A., Bessac, B. F., Sivula, M., and Jordt, S. E. (2008) TRPA1 mediates the noxious effects of natural sesquiterpene deterrents. J Biol Chem
34. Bautista, D. M., Jordt, S. E., Nikai, T., Tsuruda, P. R., Read, A. J., Poblete, J., Yamoah, E. N., Basbaum, A. I., and Julius, D. (2006) TRPA1 mediates the inflammatory actions of environmental irritants and proalgesic agents. Cell 124, 1269-1282
35. McNamara, C. R., Mandel-Brehm, J., Bautista, D. M., Siemens, J., Deranian, K. L., Zhao, M., Hayward, N.J., Chong, J. A., Julius, D., Moran, M. M., and Fanger, C. M. (2007) TRPA 1 mediates formalin-induced pain. Proc Natl Acad Sci USA 104, 13525-13530
36. Bessac, B. F., Sivula, M., von Helm, C. A., Escalera, J., Cohn, L., and Jordt, S. E. (2008) TRPA1 is a major oxidant sensor in murine airway sensory neurons. J Clin Invest 118, 1899-1910
37. Andersson, D. A., Gentry, C., Moss, S., and Bevan, S. (2008) Transient receptor potential A1 is a sensory receptor for multiple products of oxidative stress. J Neurosci 28, 2485-2494
38. Morris, J. B., Wilkie, W. S., and Shusterman, D. J. (2005) Acute respiratory responses of the mouse to chlorine. Toxicol Sci 83, 380-387
39. Andre, E., Campi, B., Materazzi, S., Trevisani, M., Amadesi, S., Massi, D., Creminon, C., Vaksman, N., Nassini, R., Civelli, M., Baraldi, P. G., Poole, D. P., Bunnett, N. W., Geppetti, P., and Patacchini, R. (2008) Cigarette smoke-induced neurogenic inflammation is mediated by alpha,beta-unsaturated aldehydes and the TRPA1 receptor in rodents. J Clin Invest
40. Nassenstein, C., Kwong, K., Taylor-Clark, T., Kollarik, M., Macglashan, D. M., Braun, A., and Undem, B. J. (2008) Expression and function of the ion channel TRPA1 in vagal afferent nerves innervating mouse lungs. J Physiol 586, 1595-1604
41. Taylor-Clark, T. E., McAlexander, M. A., Nassenstein, C., Sheardown, S. A., Wilson, S., Thornton, J., Carr, M. J., and Undem, B. J. (2008) Relative contributions of TRPA1 and TRPV1 channels in the activation of vagal bronchopulmonary C-fibres by the endogenous autocoid 4-oxononenal. JPhysiol
42. Simon, S. A., and Liedtke, W. (2008) How irritating: the role of TRPA1 in sensing cigarette smoke and aerogenic oxidants in the airways. J Clin Invest
43. Gerhold, K. A., and Bautista, D. M. (2008) TRPA1: irritant detector of the airways. J Physiol 586, 3303
44. Brone, B., Peeters, P. J., Marrannes, R., Mercken, M., Nuydens, R., Meert, T., and Gijsen, H. J. (2008) Tear gasses CN, CR, and CS are potent activators of the human TRPA1 receptor. Toxicol Appl Pharmacol
45. Vaughan, R. P., Szewczyk, M. T., Jr., Lanosa, M. J., Desesa, C. R., Gianutsos, G., and Morris, J. B. (2006) Adenosine sensory transduction pathways contribute to activation of the sensory irritation response to inspired irritant vapors. Toxicol Sci 93, 411-421
46. Hinman, A., Chuang, H. H., Bautista, D. M., and Julius, D. (2006) TRP channel activation by reversible covalent modification. Proc Natl Acad Sci USA 103, 19564-19568
47. Macpherson, L. J., Dubin, A. E., Evans, M. J., Marr, F., Schultz, P. G., Cravatt, B. F., and Patapoutian, A. (2007) Noxious compounds activate TRPA1 ion channels through covalent modification of cysteines. Nature 445, 541-545
48. Grynkiewicz, G., Poenie, M., and Tsien, R. Y. (1985) A new generation of Ca2+ indicators with greatly improved fluorescence properties. J Biol Chem 260, 3440-3450
49. Zhou, Z., and Neher, E. (1993) Mobile and immobile calcium buffers in bovine adrenal chromaffin cells. J Physiol 469, 245-273
50. Kao, J. P. (1994) Practical aspects of measuring [Ca2+] with fluorescent indicators. Methods Cell Biol 40, 155-181
51. Bessac, B. F., and Fleig, A. (2007) TRPM7 channel is sensitive to osmotic gradients in human kidney cells. J Physiol 582, 1073-1086
52. Xu, H., Delling, M., Jun, J. C., and Clapham, D. E. (2006) Oregano, thyme and clove-derived flavors and skin sensitizers activate specific TRP channels. Nat Neurosci 9, 628-635
53. Lee, S. P., Buber, M. T., Yang, Q., Cerne, R., Cortes, R. Y., Sprous, D. G., and Bryant, R. W. (2008) Thymol and related alkyl phenols activate the hTRPA1 channel. Br J Pharmacol 153, 1739-1749
54. Zurborg, S., Yurgionas, B., Jira, J. A., Caspani, O., and Heppenstall, P. A. (2007) Direct activation of the ion channel TRPA1 by Ca2+. Nat Neurosci 10, 277-279
55. Doerner, J. F., Gisselmann, G., Hatt, H., and Wetzel, C. H. (2007) Transient receptor potential channel A1 is directly gated by calcium ions. J Biol Chem 282, 13180-13189
56. Dai, Y., Wang, S., Tominaga, M., Yamamoto, S., Fukuoka, T., Higashi, T., Kobayashi, K., Obata, K., Yamanaka, H., and Noguchi, K. (2007) Sensitization of TRPA1 by PAR2 contributes to the sensation of inflammatory pain. J Clin Invest 117, 1979-1987
57. Kim, D., and Cavanaugh, E. J. (2007) Requirement of a soluble intracellular factor for activation of transient receptor potential A1 by pungent chemicals: role of inorganic polyphosphates. J Neurosci 27, 6500-6509
58. Nagata, K., Duggan, A., Kumar, G., and Garcia-Anoveros, J. (2005) Nociceptor and hair cell transducer properties of TRPA1, a channel for pain and hearing. J Neurosci 25, 4052-4061
59. Ramsey, I. S., Delling, M., and Clapham, D. E. (2006) An introduction to TRP channels. Annu Rev Physiol 68, 619-647
60. Petrus, M., Peier, A. M., Bandell, M., Hwang, S. W., Huynh, T., Olney, N., Jegla, T., and Patapoutian, A. (2007) A role of TRPA1 in mechanical hyperalgesia is revealed by pharmacological inhibition. Mol Pain 3, 40
61. Alarie, Y. (1973) Sensory irritation of the upper airways by airborne chemicals. Toxicol Appl Pharmacol 24, 279-297
62. Sangha, G. K., and Alarie, Y. (1979) Sensory irritation by toluene diisocyanate in single and repeated exposures. Toxicol Appl Pharmacol 50, 533-547
63. Morris, J. B., Stanek, J., and Gianutsos, G. (1999) Sensory nerve-mediated immediate nasal responses to inspired acrolein. J Appl Physiol 87, 1877-1886
64. Lu, S. G., Zhang, X., and Gold, M. S. (2006) Intracellular calcium regulation among subpopulations of rat dorsal root ganglion neurons. J Physiol 577, 169-190
65. Hill, A. R., Silverberg, N. B., Mayorga, D., and Baldwin, H. E. (2000) Medical hazards of the tear gas CS. A case of persistent, multrsystem, hypersensitivity reaction and review of the literature. Medicine (Baltimore) 79, 234-240
66. Hu, H., Fine, J., Epstein, P., Kelsey, K., Reynolds, P., and Walker, B. (1989) Tear gas—harassing agent or toxic chemical weapon? Jama 262, 660-663
67. Thomas, R. J., Smith, P. A., Rascona, D. A., Louthan, J. D., and Gumpert, B. (2002) Acute pulmonary effects from o-chlorobenzylidenemalonitrile “tear gas”: a unique exposure outcome unmasked by strenuous exercise after a military training event. Mil Med 167, 136-139
68. Treudler, R., Tebbe, B., Blume-Peytavi, U., Krasagakis, K., and Orfanos, C. E. (1999) Occupational contact dermatitis due to 2-chloracetophenone tear gas. Br J Dermatol 140, 531-534
69. Vaca, F. E., Myers, J. H., and Langdorf, M. (1996) Delayed pulmonary edema and bronchospasm after accidental lacrimator exposure. Am J Emerg Med 14, 402-405
70. Varma, S., and Holt, P. J. (2001) Severe cutaneous reaction to CS gas. Clin Exp Dermatol 26, 248-250
71. Goldman, L. R., Mengle, D., Epstein, D. M., Fredson, D., Kelly, K., and Jackson, R. J. (1987) Acute symptoms in persons residing near a field treated with the soil fumigants methyl bromide and chloropicrin. West J Med 147, 95-98
72. (2004) Illness associated with drift of chloropicrin soil fumigant into a residential area—Kern County, Calif., 2003. MMWR Morb Mortal Wkly Rep 53, 740-742
73. Brooks, S. M., Weiss, M. A., and Bernstein, I. L. (1985) Reactive airways dysfunction syndrome (RADS). Persistent asthma syndrome after high level irritant exposures. Chest 88, 376-384
74. Shakeri, M. S., Dick, F. D., and Ayres, J. G. (2008) Which agents cause reactive airways dysfunction syndrome (RADS)? A systematic review. Occup Med (Lond) 58, 205-211
75. Prezant, D. J., Levin, S., Kelly, K. J., and Aldrich, T. K. (2008) Upper and lower respiratory diseases after occupational and environmental disasters. Mt Sinai J Med 75, 89-100
76. Cullen, M. R. (1987) The worker with multiple chemical sensitivities: an overview. Occup Med 2, 655-661
77. Millqvist, E., Bende, M., and Lowhagen, O. (1998) Sensory hyperreactivity—a possible mechanism underlying cough and asthma-like symptoms. Allergy 53, 1208-1212

1. A method for inhibiting or alleviating the noxious effects from toxicant exposure comprising administering an effective amount of a compound that inhibits a TRPA1 function, before or after exposure thereto, wherein the compound blocks the TRPA1 receptor so as to inhibit or counter the physical effects of the chemical irritants/toxicants.

2. A method of using agents which can modulate TRPA1 function to inhibit the physical effects of chemical irritants/toxicants when given prior to exposure or to lessen the physical effects when administered post exposure.

3. A method for counteracting the acute physical noxious effects of toxicants, including but not limited to, tear gases, chlorine, hydrogen peroxide, ammonia, phosgene, chloropicrin, isocyanates, including counteracting inflammation, lachrymation, blepharospasm, respiratory irritation and depression, airway mucus secretion, airway obstruction and injury, cough and incapacitation and cutaneous chemical injuries in a subject comprising administering to said subject an effective amount of a TRPA 1 antagonist prior to exposure to said toxicants

4. (canceled)

5. A method of preventing or treating a disease or condition in a mammal, which disease or condition includes hypersensitivity to chemical stimuli, particularly in regards to inflammatory airway conditions, such as asthma and rhinitis, comprising administering to the mammal a therapeutically effective amount of a compound that inhibits TRPA1 function, wherein the compound reduces the hypersensitity and mediates the response to such chemical stimuli in the mammal.

6-7. (canceled)

8. The method of any one of claims 1-3 and 5 wherein the method comprises administering an effective amount of a compound of effective amount of a compound of Formula I or a salt thereof, or a solvate, hydrate, oxidative metabolite or prodrug of the compound or its salt: 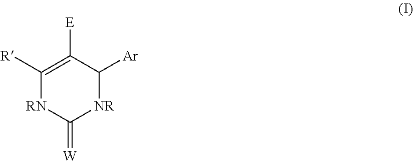
wherein W represents O or S, preferably S; R, independently for each occurrence, represents H or lower alkyl, preferably H; R′ represents substituted or unsubstituted alkyl or substituted or unsubstituted aryl; E represents carboxylic acid (CO₂H), ester or amide; and Ar represents a substituted or unsubstituted aryl ring.

9. The method of any one of claims 1-3 and 5 wherein the method comprises administering an effective amount of a compound of Formula II or a salt thereof, or a solvate, hydrate, oxidative metabolite or prodrug of the compound or its salt: 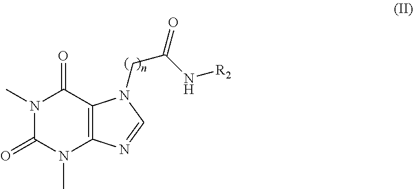
wherein n is an integer from 1 to 3; and R₂ represents a substituent, which is optionally substituted alkyl, optionally substituted aryl, optionally substituted heteroaryl, optionally substituted aralkyl, optionally substituted cycloalkyl, optionally substituted heterocyclyl, or optionally substituted heteroaralkyl.

10. The method of any one of claims 1-3 and 5 wherein the method comprises administering an effective amount of a compound of the following formula:

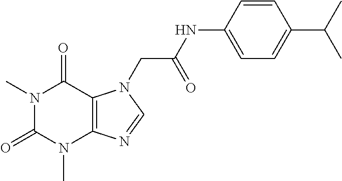

11. The method of any one of claims 1-3 and 5 wherein the method comprises administering an effective amount of a compound of Formula III or a salt thereof, or a solvate, hydrate, oxidative metabolite or prodrug of the compound or its salt: 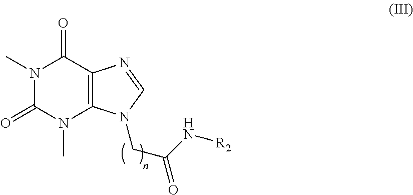
wherein n is an integer from 1 to 3; and R₂ represents a substituent, optionally substituted alkyl, optionally substituted aryl, optionally substituted heteroaryl, optionally substituted aralkyl, optionally substituted cycloalkyl, optionally substituted heterocyclyl, or optionally substituted heteroaralkyl.

12. The method of any one of claims 1-3 and 5 wherein the method comprises administering an effective amount of a compound of Formula IV or a salt thereof, or a solvate, hydrate, oxidative metabolite or prodrug of the compound or its salt: 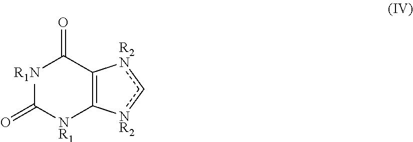
wherein R₁, independently for each occurrence, represents H or lower alkyl; one occurrence of R₂ is absent and one occurrence of R₂ is MₘR₃; R₃ represents substituted or unsubstituted aryl; M, independently for each occurrence, represents a substituted or unsubstituted methylene group (e.g., substituted with lower alkyl, oxo, hydroxyl, etc.), NR₁, O, S, S(O), or S(O₂), preferably selected such that no two heteroatoms are adjacent to each other; and m is an integer from 0-10, preferably where MₘR₃ represents: 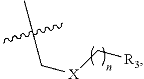
wherein n is an integer between 0 and 4; and X is or —C(═O)NR₄— wherein R₄ is H or lower alkyl, preferably —C(═O)NH—.

13. The method of any one of claims 1-3 and 5 wherein the method comprises administering an effective amount of at least one compound selected from the group consisting of 2-(1,3-dimethyl-2,6-dioxo-1,2,3,6-tetrahydro-7H-purin-7-yl)-N-(4-isopropylphenyl)acetamide, 4-(4-Chlorophenyl)-3-methylbut-3-en-2-oxime, ammoniated ruthenium oxychloride, and combinations thereof.

14. The method of any one of claims 1-3 and 5 wherein the compound that inhibits TRPA1 function is formulated for administration in a form selected from the group consisting of topically, orally, transdermally, rectally, vaginally, parentally, intranasally, intraocularly, intravenously, intramuscularly, intraarterially, intrathecally, intracapsularly, intraorbitally, intracardiacly, intradermally, intraperitoneally, transtracheally, subcutaneously, subcuticularly, intraarticularly, subcapsularly, subarachnoidly, intraspinally, intrasternally or by inhalation.

15. A kit containing a pharmaceutical preparation in a unit dosage form suitable for use in a human patient, or for veterinary use, for treating or preventing the noxious effects from exposure to a toxicant, the pharmaceutical preparation containing an effective amount of a counteracting agent which is a TRPA1 antagonist compound that inhibits a TRPA1 function, before or after exposure thereto, wherein the compound blocks the TRPA1 receptor so as to inhibit or counter the physical effects of the chemical irritants/toxicants.

16-30. (canceled)
